(19) United States
(12) Patent Application Publication
(10) Pub. No.: US 2024/0350850 A1
(43) Pub. Date: Oct. 24, 2024

(54) DEVICES, SYSTEM AND METHODS FOR EXERCISING WITH MUSCLE STIMULATION

(71) Applicants: Kristen Gelenitis, Fairview Park, OH (US); Ronald J. Triolo, Cleveland Heights, OH (US); Lisa Lombardo, Chardon, OH (US); Kevin M. Foglyano, Lakewood, OH (US); William Rasper, Kent, OH (US); Mark Nandor, Cleveland Heights, OH (US); Randolf Kobetic, Rocky River, OH (US); Roger Quinn, Akron, OH (US)

(72) Inventors: Kristen Gelenitis, Fairview Park, OH (US); Ronald J. Triolo, Cleveland Heights, OH (US); Lisa Lombardo, Chardon, OH (US); Kevin M. Foglyano, Lakewood, OH (US); William Rasper, Kent, OH (US); Mark Nandor, Cleveland Heights, OH (US); Randolf Kobetic, Rocky River, OH (US); Roger Quinn, Akron, OH (US)

(21) Appl. No.: 18/686,729

(22) PCT Filed: Aug. 26, 2022
(86) PCT No.: PCT/US2022/041664

§ 371 Date: Feb. 26, 2024

Related U.S. Application Data
(60) Provisional application No. 63/237,638, filed on Aug. 27, 2021.

(51) Int. Cl. A63B 21/00; A61G 5/04; A63B 24/00; A63B 71/06; B62J 45/20; B62J 45/413; B62J 45/415; B62J 45/416; B62M 3/08; B62M 6/50; B62M 6/65

(52) U.S. Cl. CPC A63B 21/00181; A61G 5/04; A63B 24/0087; A63B 71/0622; B62J 45/20; B62M 3/086; B62M 6/50; A63B 2213/004; A63B 2220/16; A63B 2220/40; A63B 2230/40; B62J 45/413; B62J 45/415; B62J 45/416; B62M 6/65

(57) ABSTRACT

A method includes measuring, continually or iteratively, positions of an exercise apparatus along a circuit, wherein the exercise apparatus is configured for cyclic movement along the circuit. A plurality of muscles of a user coupled to the exercise apparatus can be stimulated based on the position of the exercise apparatus. The exercise apparatus can be a cycling device or a rowing machine.

### CROSS-REFERENCE TO RELATED APPLICATION

This application claims priority to and the benefit of the filing date of U.S. Provisional Patent Application No. 63/237,638, filed Aug. 27, 2021, the entirety of which, including the appendices, is hereby incorporated by reference herein.

### FIELD

This disclosure is directed to devices, systems, and methods for exercising with functional neural stimulation.

### BACKGROUND

Exercising can be difficult for individuals with little or no control over limb muscles, such as those living with spinal cord injury, stroke, or multiple sclerosis. Some work has been done in cyclically stimulating a plurality of muscles simultaneously, but this can exhaust the muscles rapidly and can otherwise be sub-optimal. One reason that previous attempts to stimulate exercise in muscles with no feeling or control is that the user falls asleep.

### SUMMARY

Described herein, in various aspects, is method comprising measuring, continually or iteratively, positions of an exercise apparatus along a circuit, wherein the exercise apparatus is configured for cyclic movement along the circuit. A plurality of muscles of a user coupled to the exercise apparatus can be stimulated based on the position of the exercise apparatus. In exemplary configurations, the exercise apparatus can be a cycling device, a rowing machine, an elliptical trainer, or similar device.

In another aspect, a method can comprise cyclically stimulating a plurality of muscles of a user having appendages comprising distal ends (e.g., feet). The distal ends of the appendages of the user can be coupled to a crankset. Cyclically stimulating the muscles of the user can comprise beginning stimulation of each muscle of the plurality of muscles at a respective first angle of the crankset and ceasing stimulation of each muscle of the plurality of muscles at a respective second angle of the crankset.

In another aspect, a system can comprise a cycling device. The cycling device can comprise a crankset and a pair of appendage receptacles that are coupled to the crankset, wherein the pair of appendage receptacles are each configured to immobilize a respective joint of a user. A crankset angle sensor can be coupled to the crankset. The crankset angle sensor can be configured to provide an output indicative of an angle of the crankset. A controller can be in communication with the crankset angle sensor. The controller can be configured to control stimulation from an external or implanted pulse generator based at least in part on the angle of the crankset.

In another aspect, a vehicle can be movable along a surface. The vehicle can comprise a plurality of wheels. A propulsion assist system can comprise at least one battery and a motor that is operatively coupled to the at least one wheel and configured to cause rotation of at least one wheel of the plurality of wheels. A controller can be in electrical communication with the motor. At least one orientation sensor can be in communication with the controller. The at least one orientation sensor can be configured to determine a sensed orientation of the vehicle. The controller can be configured to modulate a power output of the motor based at least in part on the sensed orientation of the cycling device.

### DESCRIPTION OF THE DRAWINGS

FIG. 1 shows a system for providing functional neural stimulation to muscles of a user.

FIG. 2 shows a feedback loop for stimulating different muscles at different times along a cycle.

FIG. 3 is a block diagram of a system in accordance with embodiments disclosed herein for providing functional neural stimulation.

FIG. 4 is an exercise apparatus (depicted as a recumbent tricycle) that is configured for use with the disclosed system providing functional neural stimulation as disclosed herein. As shown, the exercise apparatus can further comprise a propulsion assistance system as disclosed herein.

FIG. 5 illustrates a carousel stimulation pattern vs. standard, conventional stimulation. Standard stimulation stimulates through multiple electrode contacts to activate multiple synergistic fiber pools each pedal stroke. Carousel patterns stimulate through single contacts at a time to activate one muscle fiber group at a time. Shown is an example carousel pattern where one contact is activated each pedal stroke during cycling.

FIG. 6 shows example carousel logic for cycling exercise. The model detects each time the cycling pedal cranks pass a certain reference angle θ using feedback from the crank angle encoder on the bike. The model alternates which contact is stimulated through and thus which subset of synergistic fibers are activated each pedal revolution each time the reference angle is passed.

FIG. 7 shows model logic for the portion of the pedal rotation in which left quadriceps are active. A carousel logic sequence identifies the end of a left quadriceps contraction by the passing of an angle outside of the L. Quad region (0=5), and switches the stimulating contact and thus fiber group used in the next contraction. Instantaneous cadence is calculated using the moving-average filtered time derivative of the crank angle and compared against a target cadence. A resulting error signal e (t) drives a PI controller to adjust PW through the active contact within each pedal stroke to maintain the target cadence. This logic is repeated in the full model for the right quadriceps, using a different crank angle (0=180) as the contact switching signal.

FIG. 8 shows a schematic diagram of an exemplary system for providing exercise with functional electric stimulation, including a perspective view of an exemplary exercise apparatus.

FIG. 9A shows an exemplary appendage receptacle. FIG. 9B shows an underside of a portion of the appendage receptacle coupled to a pedal of a crank.

FIG. 10A shows a crankset angle sensor comprising a rotary encoder and a transmission for coupling a crankset to the rotary encoder. FIG. 10B shows a block diagram of components for communicating data from the crankset angle sensor to a controller.

FIG. 11A is a perspective view of an exemplary orientation sensor. FIG. 11B is a perspective view of the orientation sensor coupled to a crankset.

FIG. 12 is a perspective view of a controller as disclosed herein.

FIG. 13 is an output of an exemplary interface for a clinician to control stimulus parameters.

FIG. 14 is a block diagram of an exemplary stimulation system as disclosed herein.

FIG. 15A is an exemplary motor as disclosed herein for providing power assistance, embodied as a hub motor. FIG. 15B is a block diagram of an exemplary system for controlling the motor.

FIG. 16 is a block diagram showing steps for providing power assistance.

FIG. 17 is a schematic diagram showing different stimulation protocols for carousel stimulation.

FIG. 18 is a chart showing work and end power data for different trials.

FIG. 19 is a chart showing power fluctuation for different trials.

FIG. 20 shows charts indicating charge accumulation over time for each stimulation condition and difference in stimulation efficiency compared with S-Max for each test condition and participant

FIG. 21 shows difference in work and end power data between controlled stimulation conditions and S-Max stimulation trials.

FIG. 22 shows Power fluctuation indices (PFI) for conventional and cadence controlled stimulation conditions.

FIG. 23 shows charts indicating charge accumulation for each controlled stimulation condition for participants with (LEFT) multiple independent stimulation channels and (RIGHT) a single stimulation channel.

FIG. 24 shows a chart indicating total charge injection for different trials.

FIG. 25 shows a chart indicating stimulation efficiency for different trials.

FIG. 26 shows mean muscle oxygenation (SmO₂) throughout S-Max and S-Cont cycling trials for certain individuals. Shaded regions represent standard deviations.

FIG. 27 shows chart indicating heart rate responses for an individual during S-Max and S-Cont stimulation-induced cycling bouts.

FIG. 28 is a block diagram of an environment comprising an exemplary computing device for controlling and interfacing with various parameters as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a sensor” can refer to one or more of such sensors.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “approximately,” “substantially,” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

The following description supplies specific details in order to provide a thorough understanding. Nevertheless, the skilled artisan would understand that the apparatus and associated methods of using the apparatus can be implemented and used without employing these specific details. Indeed, the apparatus and associated methods can be placed into practice by modifying the illustrated apparatus and associated methods and can be used in conjunction with any other apparatus and techniques conventionally used in the industry.

### Exercise Apparatus with Functional Neural Stimulation

### Introduction

Functional neural stimulation (FNS) can be used to exercise certain muscles and the associated cardiovascular system. An FNS system can comprise one or more electrodes and a pulse generator in communication with the electrode(s). The electrodes can provide stimuli to muscles to activate said muscles.

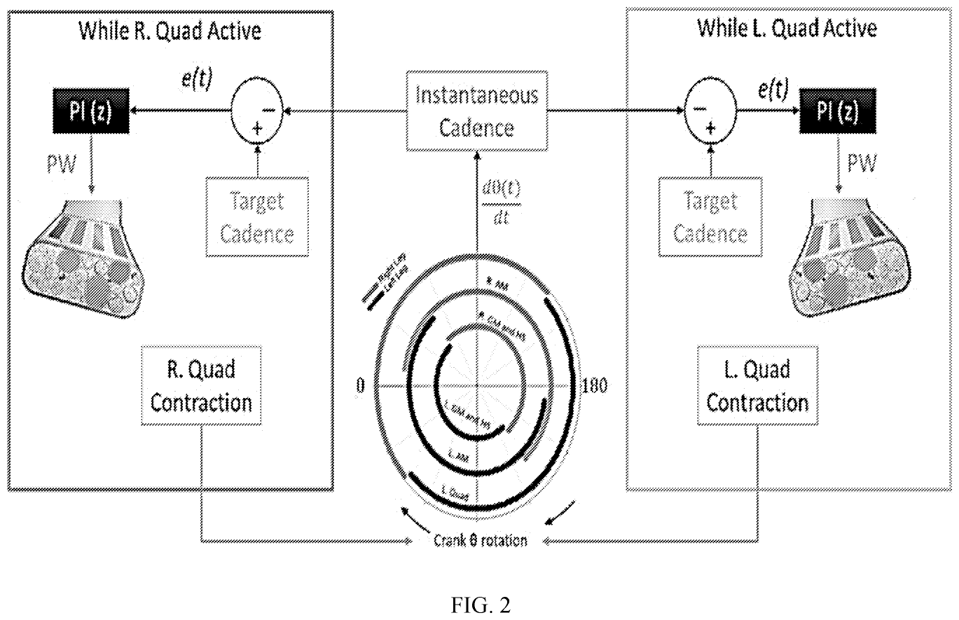

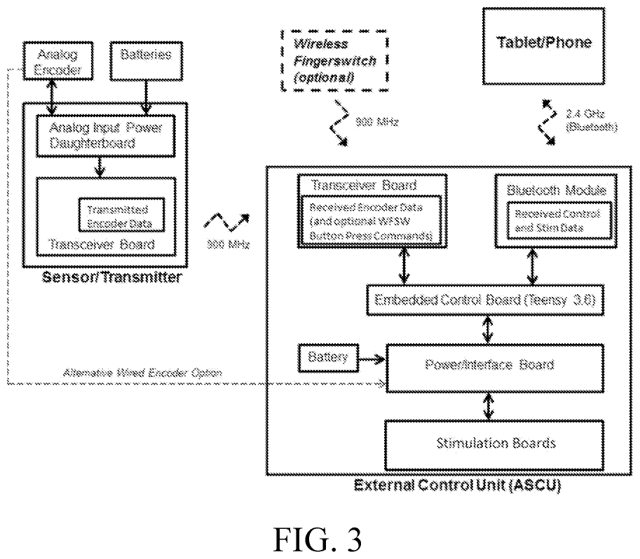

Referring to FIG. 2, it is contemplated that it can be desirable to activate muscles in a particular sequence in order to achieve optimal results. For example, simultaneous activation of antagonistic muscles can cause them to work against each other, thereby rapidly fatiguing the muscles and not allowing sufficient blood flow or effective cardiovascular exercise. Simultaneous activation of non-antagonistic or synergistic muscles can result in stronger output if the nerve fibers do not overlap. Accordingly, non-simultaneously activating different muscles on each side of the body or rotating between synergistic muscles on the same side can have advantageous exercise results.

According to some aspects, a user can be coupled to an exercise apparatus. For example, the user can strap her feet into appendage receptacles of a stationary bike, a recumbent tricycle, or other cycling device. As can be understood, the user need not have feet. For example, the appendage receptacles can be configured to receive distal portions of the appendages of an amputee having intact lower motor nerves to her leg and hip muscles. In further aspects, the user can be strapped to a rowing machine with her feet (or other appendages) attached to a foot pad, and her thighs and/or waist can be attached to a seat that is movable relative to the foot pad. The exercise apparatus can be configured for cyclic movement along a circuit. The circuit can be a path that starts and ends in the same place. For example, a circuit of a cycling device can be a revolution of the crankset. A circuit of the rowing machine can be one outward and inward movement of the seat relative to the foot pad. In further aspects, the exercise apparatus can be configured to be used with any user. For example, various other exercise apparatuses are contemplated, including a crank shaft configured to be operated with the upper limbs or neck of a user to improve muscles and respiratory function. Other apparatuses consistent with the scope of this disclosure are contemplated that exercise the user via stepping motions (e.g., a stair stepper), climbing motions (e.g., optionally, for upper limbs), bicep curls, etc.

According to some aspects, the exercise apparatus can comprise a sensor that is configured to determine the position of the exercise apparatus along the circuit. The sensor can be in communication with a controller. The controller can continuously or iteratively monitor positions of the exercise apparatus along the circuit.

The controller can be configured to cause functional neural stimulation of a plurality of muscles. The muscles (on each side (e.g., the left and right sides) of the user) can be stimulated non-simultaneously at different positions (e.g., beginning and ending positions) along the circuit. For example, the left quadriceps, the left gluteus maximus, the left hamstring extensor, the left hamstring flexor, the right quadriceps, the right gluteus maximus, the right hamstring extensor, and the right hamstring flexor can all be stimulated to flex and release at different start and stop positions along the circuit. In further aspects, the left and/or right tibialis anterior can be stimulated. In yet further aspects, the lumbar trunk extensors can be stimulated. Various other muscles (and combinations thereof) can be stimulated based on the application. In yet further aspects, a flexion withdrawal reflex can be elicited from a sensory nerve. The muscles selected and the timing and intensity of the stimulating currents delivered can be customized for the desired motion along the circuit.

Optionally, the positions at which the different muscles begin to receive stimulation and stop receiving stimulation can be adjusted. This can be to achieve a target speed of the circuit, a target heart rate, or other desired metric. The adjustment can be performed manually by a clinician or by the user. In further aspects, the adjustments can be performed by an algorithm (e.g., a machine learning algorithm).

In some aspects, the position of the exercise apparatus can be measured by a sensor. For example, a rotary encoder can be coupled to the crankset of the cycling device. In further aspects, an inertial measurement unit can be affixed to the crankset. In further aspects, an inertial measurement unit can be affixed to the legs of the user to determine joint angles. The sensor(s) can optionally be in wireless communication with the controller.

Embodiments disclosed herein can advantageously improve muscle and respiratory function of the user.

### Exemplary Exercise Apparatuses

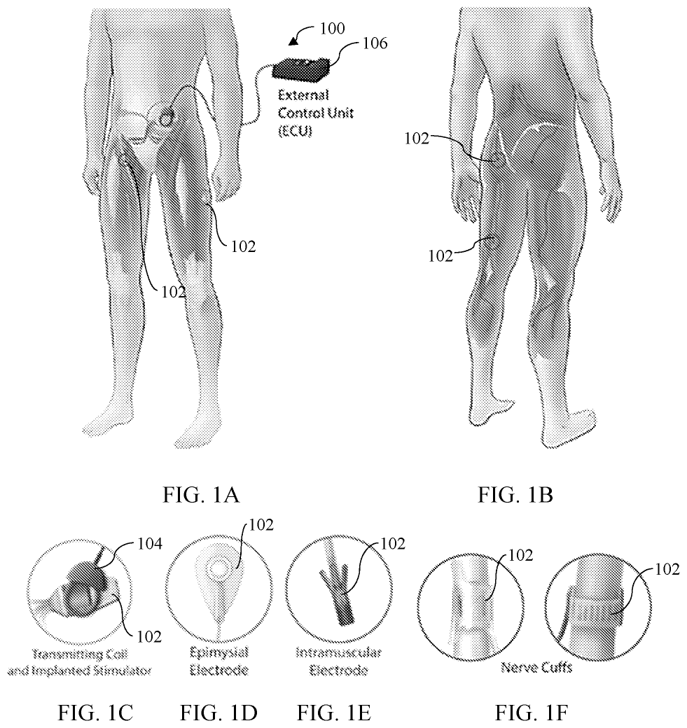

FIGS. 1A and 1B illustrate a system 100 for providing functional neural stimulation to a user. A pulse generator 106 can be configured to actuate the electrodes 102. The pulse generator 106 can optionally be an independent pulse generator.

Electrodes 102 can be operatively positioned for stimulating nerves. FIGS. 1C-1F illustrate exemplary electrodes 102 of the system 100. For example, the electrodes 102 can be embodied as nerve cuffs, intramuscular electrodes, epimysial electrodes, or implanted stimulators. In aspects in which the electrodes 102 comprise one or more implanted stimulators, the system 100 can comprise one or more transmitting coils 104 that remotely activate the implanted stimulators via induction. In other aspects, electrical current can be delivered to the nerves via electrodes adhered to the skin or embedded at the proper locations in tight fitting garments (not pictured). It is contemplated that the nerves can be stimulated to cause certain muscles, or portions thereof, to contract, thereby exercising said muscles and the associated cardiovascular system.

Referring to FIG. 8, an exercise system 200 can comprise an exercise apparatus 300. In some optional aspects, the exercise apparatus 300 can be a cycling device. In other aspects, the exercise apparatus 300 can be a rowing machine. In some aspects, the exercise apparatus can be a stair stepper, a device for performing climbing motions (e.g., optionally, for upper limbs), or a device for performing bicep curls and/or extensions.

Referring also to FIGS. 10A and 10B, in aspects in which the exercise apparatus 300 is a cycling device, the cycling device can comprise a crankset 302. A pair of appendage receptacles 304 can be coupled to the crankset 302. The appendage receptacles 304 can be, for example, boots that are configured to receive feet or distal portions of legs of a user. In other aspects, the appendage receptacles 304 can be configured to receive hands or distal portions of arms of a user. In some optional aspects, the pair of appendage receptacles 304 can each be configured to immobilize a respective joint of a user. For example, the appendage receptacles 304 can be configured to immobilize the ankle of the user.

A crankset angle sensor 310 can be coupled to the crankset 302. The crankset angle sensor 310 can be configured to provide an output indicative of an angle of the crankset 302.

A controller 320 can be in communication with the crankset angle sensor 310. The controller 320 can be configured to control stimulation from an external or implanted pulse generator based at least in part on the angle of the crankset. For example, the controller 320 can be in operative communication with the pulse generator 106 of the FNS system 100, and the controller 320 can cause the pulse generator 106 to deliver current to the electrodes.

Optionally, the controller 320 can comprise a wireless receiver 322. The cycling device can further comprise a wireless transmitter 324 that is in communication with the crankset angle sensor 310. The controller 320 can be in wireless communication with the crankset angle sensor 310 by the wireless transmitter 324. In alternative aspects, the controller 320 can be in wired communication with the crankset angle sensor 310.

In some aspects, the crankset angle sensor 310 can comprise a rotary encoder 330. The exercise system 200 can further comprise a transmission 332 that couples the crankset angle sensor (e.g., the rotary encoder) to the crankset. In some aspects, the transmission 332 can comprise a direct coupling between the rotary encoder and the crankset. In some aspects, the transmission 332 can comprise a first pulley 334 that is rotationally fixed to the crankset, a second pulley 336 that is coupled to the rotary encoder, and a belt 338 that extends between the first pulley and the second pulley. In some aspects, the transmission 332 can comprise a first gear that is coupled to the rotary encoder and a second gear that is coupled to the crankset, wherein the first gear is coupled to the second gear.

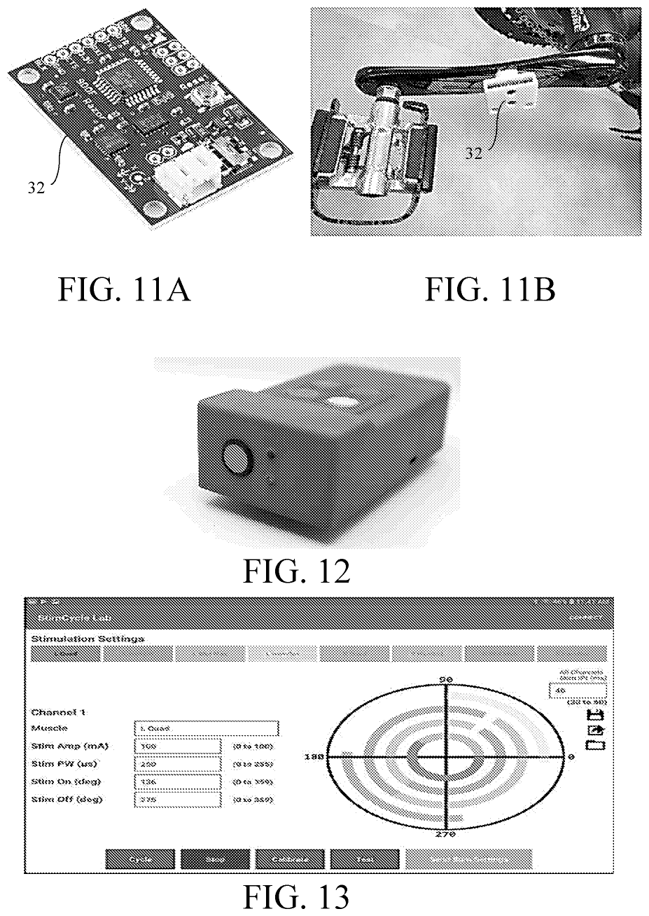

In some aspects, and as illustrated in FIG. 11B, the crankset angle sensor 310 can comprise at least one orientation sensor that is coupled to the crankset 310. The at least one orientation sensor can comprise an inertial measurement unit. Optionally, the exercise system 200 can comprise a plurality of orientation sensors.

In some aspects, the pulse generator 106 in communication with the controller 320 can be an external pulse generator. In some aspects, the pulse generator 106 in communication with the controller 320 can be an implanted pulse generator.

The exercise system 200 can comprise the plurality of electrodes 102 that are configured to stimulate respective muscles of the user.

Referring to FIG. 2, the controller can be configured to deliver functional neural stimulation to a plurality of groups of muscle fibers. For each group of muscle fibers, stimulation is configured to start at a respective first rotational position of the crankset and cease at a respective second rotational position of the crankset 302. In this way, the groups of muscle fibers can work cooperatively to turn the crankset 302 without resisting each other. Further, the groups of muscle fibers can contract during ideal positions of the crankset 302 to synergistically drive the crankset when the groups of muscles are delivering the best mechanical advantage to the crankset.

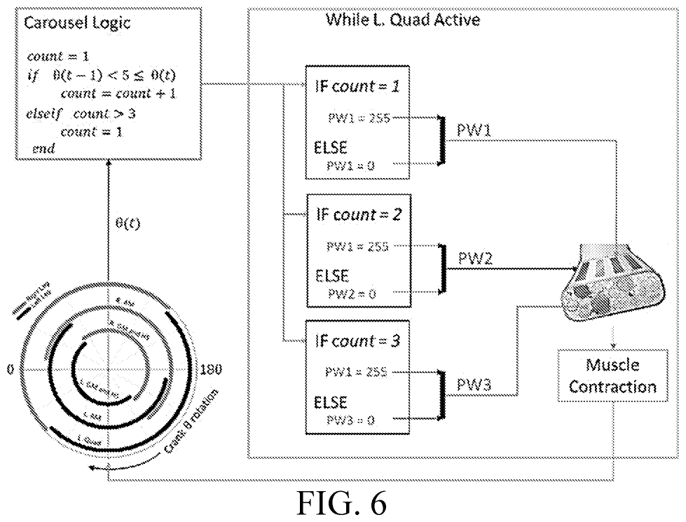

Still further, in some aspects and with reference to FIGS. 5-6, for sequential rotations of the crankset 302, different portions of a muscle can be stimulated. For example, for a first rotation, a first portion of a muscle (e.g., a left quadriceps) can be stimulated during a portion of the first rotation, and a second portion of the muscle can be stimulated during the same portion of the next sequential rotation. In still further aspects, a third portion of the muscle can be stimulated during the same portion of the following sequential rotation. In this way, portions of the muscle have time to rest in order to inhibit undesirably fatiguing the muscle. Such stimulation for sequential cycles is referred to herein as “carousel stimulation.”

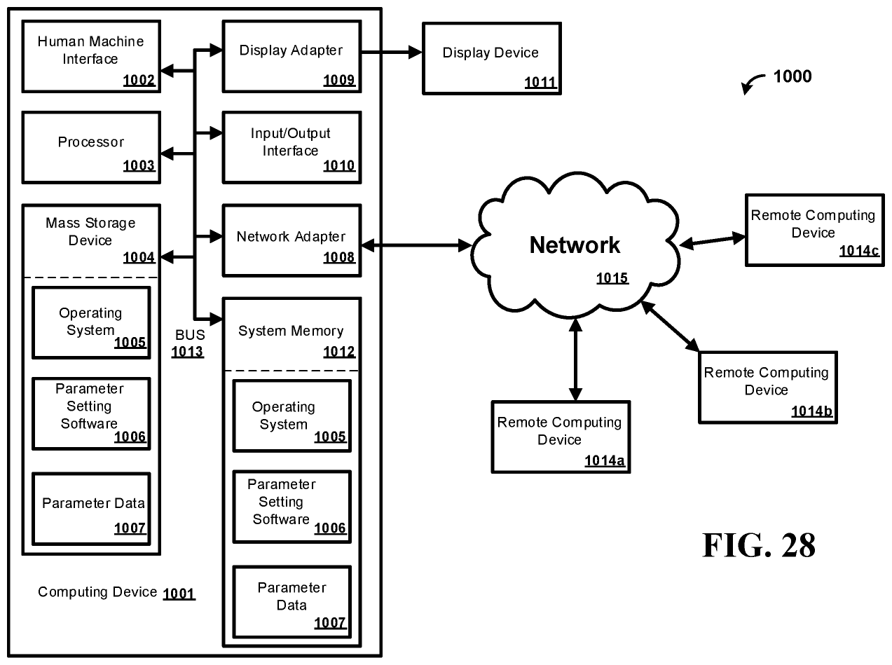

In some aspects, and as shown in FIG. 28, the exercise system 200 can comprise a computing device 1001 in communication with the controller 320. The computing device 1001 can be configured to provide an interface to a clinician. The computing device 1001 can receive, by the interface, at least one parameter selection from the clinician and set at least one control parameter of the controller. For example, the at least one control parameter can comprise at least one of a stimulation current, a pulse width, a start angle corresponding to an angle of the crankset at which stimulation begins, or a stop angle corresponding to an angle of the crankset at which stimulation ceases. Each control parameter of the at least one control parameter can be associated with a particular group of fibers of muscle fibers or a particular muscle.

Referring to FIG. 8, in some aspects, the exercise apparatus can comprise a plurality of wheels 340. The crankset 302 can be coupled to at least one wheel of the plurality of wheels. For example, in some aspects, and as illustrated, the cycling device can be a recumbent tricycle.

In alternative aspects, the cycling device can be a stationary bike. In some optional aspects, the exercise system 200 can comprise a display that is configured to display visual feedback associated with use of the cycling device. For example, the display can show speed, power, calories burned, and/or any information associated with use of the exercise vehicle. In further aspects, the display can show a simulated view, such as that of a user biking down a path. In some optional aspects, the display can be a virtual reality device or an augmented reality device. Accordingly, in exemplary aspects, the display can comprise goggles. It is contemplated that such visual feedback can keep a user engaged.

In some optional aspects, the exercise system 200 can comprise at least one respiration measurement device (e.g., a flow meter). In some optional aspects, the exercise system 200 can comprise at least one grip sensor.

A method can comprise cyclically stimulating fibers of a plurality of muscles of a user having at least one appendage comprising a distal portion. The distal portion of the at least one appendage of the user can be coupled to a crankset. Cyclically stimulating the fibers of the plurality of muscles of the user can comprise beginning stimulation of the fibers of each muscle of the plurality of muscles at a respective first angle of the crankset and ceasing stimulation of the fibers of each muscle of the plurality of muscles at a respective second angle of the crankset.

In some aspects, the plurality of muscles can comprise two or more of: a left quadriceps, a left gluteus maximus, a left hamstring extensor, a left hamstring flexor, a right quadriceps, a right gluteus maximus, a right hamstring extensor, or a right hamstring flexor. In further aspects, the plurality of muscles can comprise each of: the left quadriceps, the left gluteus maximus, the left hamstring extensor, the left hamstring flexor, the right quadriceps, the right gluteus maximus, the right hamstring extensor, and the right hamstring flexor.

Still further, in some aspects and with reference to FIGS. 5-6, for sequential rotations of the crankset, different portions of a muscle can be stimulated. For example, for a first rotation, a first portion of a muscle (e.g., a left quadriceps) can be stimulated during a portion of the first rotation, and a second portion of the muscle can be stimulated during the same portion of the next sequential rotation. In still further aspects, a third portion of the muscle can be stimulated during the same portion of the following sequential rotation. In this way, portions of the muscle have time to rest in order to inhibit undesirably fatiguing the muscle.

The method can further comprise measuring an exercise metric, comparing the exercise metric to a target exercise metric, and modifying at least one stimulation parameter based on the exercise metric. In some exemplary aspects, the exercise metric can be a heart rate, and the target exercise metric can be a target heart rate. In some exemplary aspects, the exercise metric can be a ventilation rate and the target exercise metric can be a target ventilation rate. In some exemplary aspects, the exercise metric can be a power output, and the target exercise metric can be a target power output. In some exemplary aspects, the exercise metric can be a crankset rotation speed, and the target exercise metric can be a target crankset rotation speed.

Modifying the at least one stimulation parameter can comprise increasing or decreasing at least one parameter to increase or decrease the exercise metric toward the target exercise metric. The at least one stimulation parameter can comprise at least one of a pulse width, a stimulation current, an angle of the crankset at which stimulation begins, or a stop angle corresponding to an angle of the crankset at which stimulation ceases.

Optionally, modifying the at least one stimulation parameter can comprise modifying the at least one parameter based on machine learning. The machine learning can comprise one of iterative learning control or reinforcement learning control.

In some aspects, the target exercise metric can be received from a clinician or the user. The target exercise metric can be received during an exercise session.

At least one visual element associated with exercise generated by stimulation of the fibers of the plurality of muscles can be displayed on a display. Optionally, the display can be, for example, an augmented reality display or a virtual reality display. In other aspects, the display can be any suitable display.

In some aspects, a volitional effort input can be received from the user. A metric associated with the volitional effort input can be displayed on the display. The volitional effort input can be, for example, a force or pressure sensor associated with grip.

In some aspects, electromyography signals of the user can be measured.

In some aspects, respiration of the user can be measured. The measured respiration can be displayed.

In some aspects, the crankset of the disclosed method can be the crankset of a stationary bike. In other aspects, crankset of the disclosed method can be the crankset of a cycling device (e.g., a recumbent tricycle) comprising a plurality of wheels, and the crankset can be coupled to at least one wheel of the plurality of wheels.

A method can comprise measuring, continually or iteratively, positions of an exercise apparatus along a circuit, wherein the exercise apparatus is configured for cyclic movement along the circuit. A plurality of muscles of a user coupled to the exercise apparatus can be cyclically stimulated based on the position of the exercise apparatus.

In some aspects, and as further disclosed herein, the exercise apparatus can be a cycling device. In some aspects, the exercise apparatus can be a stationary bike. In some aspects, the exercise apparatus can be an elliptical trainer. In some aspects, the exercise apparatus can be a rowing machine. In these aspects, measuring, continually or iteratively, the positions of the exercise apparatus along the circuit comprises using a linear position sensor to measure the positions of the exercise apparatus along the circuit.

The method can further comprise measuring an exercise metric, comparing the exercise metric to a target exercise metric, and modifying at least one stimulation parameter based on the exercise metric.

As further disclosed herein, the exercise metric can be a heart rate, a ventilation rate, a power output, or a circuit completion speed. Modifying the at least one stimulation parameter comprises increasing or decreasing at least one parameter to increase or decrease the exercise metric toward the target exercise metric. The at least one stimulation parameter can comprise at least one of a pulse width, a stimulation current, a position of the exercise apparatus along the circuit at which stimulation begins, or position of the exercise apparatus along the circuit at which stimulation ceases.

Optionally, modifying the at least one stimulation parameter can comprise modifying the at least one parameter based on machine learning. The machine learning can comprise one of iterative learning control or reinforcement learning control. More particularly, a computing device as disclosed herein can comprise or be communicatively coupled to a machine learning model as further disclosed below. It is contemplated that the machine learning model can analyze one or more exercise metrics and determine optimal settings for one or more stimulation parameters.

In some aspects, the target exercise metric can be received from a clinician or the user. The target exercise metric can be received during an exercise session.

At least one visual element associated with exercise generated by stimulation of the fibers of the plurality of muscles can be displayed on a display. Optionally, the display can be, for example, an augmented reality display or a virtual reality display. In other aspects, the display can be any suitable display.

In some aspects, a volitional effort input can be received from the user. A metric associated with the volitional effort input can be displayed on the display. The volitional effort input can be, for example, a force or pressure sensor associated with grip.

In some aspects, electromyography signals of the user can be measured.

In some aspects, respiration of the user can be measured. The measured respiration can be displayed.

### Exemplary Machine Learning Features

In exemplary aspects, and as further disclosed herein, the systems and apparatuses disclosed herein can operate on a network, which can facilitate communication between each device/entity of the system. The network may be an optical fiber network, a coaxial cable network, a hybrid fiber-coaxial network, a wireless network, a satellite system, a direct broadcast system, an Ethernet network, a high-definition multimedia interface network, a Universal Serial Bus (USB) network, or any combination thereof. Data may be sent/received via the network by any device/entity of the system via a variety of transmission paths, including wireless paths (e.g., satellite paths, Wi-Fi paths, cellular paths, etc.) and terrestrial paths (e.g., wired paths, a direct feed source via a direct line, etc.).

In exemplary aspects, the network can comprise a server, which may be a single computing device or a plurality of computing devices. For purposes of explanation, the description herein will describe the server and the computing device 1001 (and remote computing device 1014a,b,c) as being separate entities with separate functions. However, it is to be understood that any data sent/received by, as well as any functions performed by, the server may apply equally to the computing device 1001 (or remote computing device 1014a,b,c)—and vice-versa. For example, the server may be a module/component of the computing device 1001—or vice-versa. Additionally, other computing devices may perform part of the functions described herein with respect to the system and apparatus.

In exemplary aspects, the server may include a storage module and a machine learning module. The computing device 1001 may be in communication with the server. For purposes of explanation, the description herein will refer to the server-specifically, the machine learning module—as the device that analyzes the exercise metrics (and other related user data); however, is to be understood that the computing device 1001 (or remote computing device 1014a,b,c) may analyze the exercise metrics and other user data in a similar manner.

As described herein, the computing device 1001 may send (e.g., upload) the exercise metrics to the server via the network. Similarly, it is contemplated that the computing device 1001 and/or remote computing device 1014a,b,c can send historical exercise metrics and/or user information to the server via the network. The machine learning module of the server may analyze the exercise metrics and historical exercise metrics and/or user information. The exercise metrics can be indicative of a current performance and/or condition of a given user. The historical exercise metrics can include previous performance and/or condition of the same user or of other users that are known to have identified properties or characteristics (such as properties or characteristics that are shared with the current user).

The machine learning module may use, as an example, a segmentation model to compare the current exercise metrics with historical exercise metrics. For example, the machine learning model may use a segmentation model to classify current exercise metrics as corresponding to or not corresponding to particular historical exercise metrics. As another example, the machine learning module can use the segmentation model to classify respective historical exercise metrics as corresponding to or not corresponding to current exercise metrics. Thus, the segmentation model may determine the level of relatedness or correlation between the current exercise metrics and the historical exercise metrics—or vice-versa.

Additionally, or alternatively, the machine learning model may be trained, as further discussed herein, by applying one or more machine learning models and/or algorithms to a plurality of training exercise metrics and user data. The term “segmentation” refers to analysis of exercise metrics and/or historical exercise metrics to determine the level of relatedness or correlation between metrics. In some cases, segmentation may be based on semantic content of the exercise metrics. For example, segmentation analysis performed on the exercise metrics may indicate particular metrics that are indicative of a particular attribute of the user. In some cases, segmentation analysis may produce segmentation data. The segmentation data may indicate one or more segments (sets) of exercise metrics among a larger group of analyzed exercise metrics. For example, the segmentation data may include a set of labels, such as pairwise labels (e.g., labels having a value indicating “yes” or “no”) indicating whether a given exercise metric corresponds to a historical exercise metric or is indicative of a particular attribute of the user (or user's performance). In some cases, labels may have multiple available values, such as a set of labels indicating whether a metric is indicative of a first attribute, a second attribute, a combination of attributes, and so on. The segmentation data may include numerical data, such as data indicating a probability that a given metric is indicative of a particular attribute(s) of the user (or user's performance). In some cases, the segmentation data may include additional types of data, such as text, database records, or additional data types, or structures.

In some examples, physical data associated with the user may be determined. The physical data may comprise—or be indicative of—one or more physical conditions or characteristics associated with the user. In exemplary aspects, the storage module may provide/send a first set of exercise metrics to the machine learning module. The machine learning module may use the segmentation model to align individual exercise metrics or sets of exercise metrics of a current user with a historical exercise metric or historical set of exercise metrics for the same user. The machine learning module may generate an output indicative of the changes relative to the historical exercise metrics.

The machine learning module may send the output to the storage module. The storage module may send the output to the computing device 1001. The computing device 1001 may receive the output image via an application, which may be displayed via a user interface of the application at the computing device 1001. A user of the application may interact with the output and provide one or more user edits, such as by adjusting an attribute/feature, modifying an attribute/feature, etc. The application may provide an indication of the one or more user edits to the server (e.g., an edited version of the output). The indication of the one or more user edits may be stored at the storage module.

Optionally, the user interface may display an output containing a listing or display of one or more attributes associated with the user or the user's performance.

As described herein, the user interface may include a plurality of editing tools that facilitate the user interacting with the output and/or the segmentation model. The user may interact with the output and/or the segmentation model and provide one or more user edits, such as by adjusting an attribute (e.g., an indication of a condition), modifying an attribute, etc. For example, the user interface may include a list of attribute categories that allow the user to categorize one or more user-defined attributes, such as particular physical conditions. The user may also modify and/or delete any attribute indicated by the segmentation model.

The application may provide an indication of one or more user edits made to any of the attributes indicated by the segmentation model (or any created or deleted attributes) to the server. For example, the application may send the indication of the one or more user edits (e.g., an edited version of the output) to the server. Expert annotation may be provided to the server by a third-party computing device. The expert annotation may be associated with the one or more user edits. For example, the expert annotation may comprise an indication of an acceptance of the one or more user edits, a rejection of the one or more user edits, or an adjustment to the one or more user edits. The one or more user edits and/or the expert annotation may be used by the machine learning module to optimize the segmentation model. For example, the one or more user edits and/or the expert annotation may be used by the machine learning module to retrain the segmentation model.

A training system may be configured to use machine learning techniques to train, based on an analysis of one or more training data sets by a training module, at least one machine learning-based classifier that is configured to classify exercise metrics of a current user as corresponding to (or being indicative of) or not corresponding to (or not being indicative of) particular attribute(s). The at least one machine learning-based classifier may comprise the machine learning module.

The training system may determine (e.g., access, receive, retrieve, etc.) the training data set. The training data set may comprise first sets of exercise metrics (e.g., a portion of a plurality of exercise metrics) associated with a plurality of users. The training system may determine (e.g., access, receive, retrieve, etc.) a second training data set, which may comprise second sets of exercise metrics (e.g., a portion of the plurality of exercise metrics) associated with the plurality of users. The first training data set and the second training data set may each contain one or more result datasets associated with exercise metrics, and each result dataset may be associated with one or more user (or user performance) attributes. Each result dataset may include a labeled list of results. The labels may comprise “attribute metric” (corresponding to a metric that indicates a particular attribute) and “non-attribute metric” (corresponding to a metric that does not indicate a particular attribute).

Exercise metric data may be randomly assigned to the training data set or to a testing data set. In some implementations, the assignment of data to a training data set or a testing data set may not be completely random. In this case, one or more criteria may be used during the assignment, such as ensuring that similar numbers of exercise metrics are in each of the training and testing data sets. In general, any suitable method may be used to assign the data to the training or testing data sets, while ensuring that the distributions of sufficient quality and insufficient quality labels are somewhat similar in the training data set and the testing data set.

The training module may train the machine learning-based classifier by extracting a feature set from the training data set according to one or more feature selection techniques. The training module may further define the feature set obtained from the training data set by applying one or more feature selection techniques to the training data set that includes statistically significant features of positive examples (e.g., metrics indicating a particular attribute(s) of a historical user) and statistically significant features of negative examples (e.g., metrics not indicating a particular attribute(s) of a historical user). The feature set extracted from the training data set and/or the training dataset may comprise segmentation data as described herein. For example, the feature set may comprise features associated with metrics that are indicative of the one or more conditions or attributes described herein. The feature set may be derived from the segmentation data indicated by the exercise metrics described herein.

The training module may extract the feature set from either of the training data sets in a variety of ways. The training module may perform feature extraction multiple times, each time using a different feature-extraction technique. In an embodiment, the feature sets generated using the different techniques may each be used to generate different machine learning-based classification models. For example, the feature set with the highest quality metrics may be selected for use in training. The training module may use the feature set(s) to build one or more machine learning-based classification models that are configured to indicate whether or not new exercise metrics are indicative of particular attribute(s) of the current user.

One or both of the training data sets may be analyzed to determine any dependencies, associations, and/or correlations between extracted features and the sufficient quality/insufficient quality labels in the training data set(s). The identified correlations may have the form of a list of features that are associated with labels for metrics indicating a particular attribute(s) of a corresponding user and labels for metrics not indicating the particular attribute(s) of the corresponding user. The features may be considered as variables in the machine learning context. The term “feature,” as used herein, may refer to any characteristic of an item of data that may be used to determine whether the item of data falls within one or more specific categories. By way of example, the features described herein may comprise the values associated with the exercise metrics disclosed herein, as well as values reflecting a comparison between current exercise metrics and historical exercise metrics.

A feature selection technique may comprise one or more feature selection rules. The one or more feature selection rules may comprise an exercise metric value and an exercise metric value occurrence rule. The exercise metric attribute occurrence rule may comprise determining which exercise metric attributes in the training data set occur over a threshold number of times and identifying those exercise metric attributes that satisfy the threshold as candidate features. For example, any exercise metric attributes that appear greater than or equal to 8 times in the training data set may be considered as candidate features. Any exercise metric attributes appearing less than 8 times may be excluded from consideration as a feature. Any threshold amount may be used as needed.

A single feature selection rule may be applied to select features or multiple feature selection rules may be applied to select features. The feature selection rules may be applied in a cascading fashion, with the feature selection rules being applied in a specific order and applied to the results of the previous rule. For example, the exercise metric attribute occurrence rule may be applied to the training data set to generate a first list of exercise metric attributes. A final list of candidate features may be analyzed according to additional feature selection techniques to determine one or more candidate groups (e.g., groups of pixel attributes). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., exercise metrics that indicate or do not indicate a particular attribute(s) of a corresponding user).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature groups. Forward feature selection is an iterative method that begins with no features in the machine learning model. In each iteration, the feature which best improves the model is added until an addition of a new feature does not improve the performance of the machine learning model. In an embodiment, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module has generated a feature set(s), the training module may generate a machine learning-based classification model based on the feature set(s). A machine learning-based classification model may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

The training module may use the feature sets extracted from one or both of the training data sets to build a machine learning-based classification model for each classification category (e.g., each attribute of a corresponding user). In some examples, the machine learning-based classification models may be combined into a single machine learning-based classification model. Similarly, the machine learning-based classifier may represent a single classifier containing a single or a plurality of machine learning-based classification models 1350 and/or multiple classifiers containing a single or a plurality of machine learning-based classification models.

The extracted features (e.g., one or more exercise metric attributes) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier may comprise a decision rule or a mapping for each candidate exercise metric attribute to assign a metric to a class (e.g., indicating or not indicating a particular attribute(s) of a corresponding user).

The candidate exercise metric attributes and the machine learning-based classifier may be used to predict a label (e.g., indicating or not indicating a particular attribute(s) of a corresponding user) for results in the testing data set (e.g., in a portion of second set of exercise metrics). In one example, the prediction for each result in the testing data set includes a confidence level that corresponds to a likelihood or a probability that the corresponding metric(s) indicates or does not indicate a particular attribute(s) of a corresponding user. The confidence level may be a value between zero and one, and it may represent a likelihood that the corresponding metric(s) belongs to a particular class. In one example, when there are two statuses (e.g., indicating or not indicating a particular attribute(s) of a corresponding user), the confidence level may correspond to a value p, which refers to a likelihood that a particular metric belongs to the first status (e.g., indicating the particular attribute(s)). In this case, the value 1-p may refer to a likelihood that the particular metric belongs to the second status (e.g., not indicating the particular attribute(s)). In general, multiple confidence levels may be provided for each metric and for each candidate metric attribute when there are more than two statuses. A top performing candidate metric attribute may be determined by comparing the result obtained for each metric with the known sufficient quality/insufficient quality status for each corresponding set of exercise metrics in the testing data set (e.g., by comparing the result obtained for each metric with the labeled metrics of the second portion of the exercise metrics). In general, the top performing candidate metric attribute for a particular attribute(s) of the corresponding user will have results that closely match the known depicting/not depicting statuses.

The top performing exercise metric attribute may be used to predict the indicating/not indicating of exercise metrics of a new/current user. For example, a new set of exercise metrics may be determined/received. The new set of exercise metrics may be provided to the machine learning-based classifier which may, based on the top performing exercise metric attribute for the particular attribute(s) of the corresponding user, classify the exercise metrics of the new set of exercise metrics as indicating or not indicating the particular attribute(s).

As noted above, the application may provide an indication of one or more user edits made to any of the attributes indicated by the segmentation model (or any created or deleted attributes) to the server. For example, the user may edit any of the attributes indicated by the segmentation model to modify boundaries of the attribute(s). Other input devices or methods of obtaining user commands may also be used. The one or more user edits may be used by the machine learning module to optimize the segmentation model. For example, the training module may extract one or more features from outputs containing one or more user edits as discussed above. The training module may use the one or more features to retrain the machine learning-based classifier and thereby continually improve results provided by the machine learning-based classifier.

A training method may be used for generating the machine learning-based classifier using the training module. The training module can implement supervised, unsupervised, and/or semi-supervised (e.g., reinforcement based) machine learning-based classification models.

The training method may determine (e.g., access, receive, retrieve, etc.) first exercise metrics associated with a plurality of historical users (e.g., first users) and second exercise metrics associated with the plurality of historical users (e.g., second users). The first exercise metrics and the second exercise metrics may each contain one or more exercise metric result datasets associated with users, and each result dataset may be associated with one or more exercise metric attributes. Each result dataset may include a labeled list of results. The labels may comprise “attribute exercise metric” and “non-attribute exercise metric.”

The training method may generate a training data set and a testing data set. The training data set and the testing data set may be generated by randomly assigning labeled exercise metric results to either the training data set or the testing data set. In some implementations, the assignment of labeled exercise metric results as training or test samples may not be completely random. In an embodiment, only the labeled exercise metric results for a specific category of user (e.g., exercise metrics for users having particular age, size, and or physical condition characteristics) may be used to generate the training data set and the testing data set. In an embodiment, a majority of the labeled exercise metric results for the specific user category may be used to generate the training data set. For example, 75% of the labeled exercise metric results for the specific category of user may be used to generate the training data set and 25% may be used to generate the testing data set.

The training method may determine (e.g., extract, select, etc.) one or more features that can be used by, for example, a classifier to differentiate among different classifications (e.g., “attribute exercise metric” vs. “non-attribute exercise metric.”). The one or more features may comprise a set of one or more exercise metric attributes. In an embodiment, the training method may determine a set of features from the first exercise metrics. In another embodiment, the training method may determine a set of features from the second exercise metrics. In a further embodiment, a set of features may be determined from labeled exercise metric results from a user category that is different than the user category associated with the labeled exercise metric results of the training data set and the testing data set. In other words, labeled exercise metric results from the different user category may be used for feature determination, rather than for training a machine learning model. The training data set may be used in conjunction with the labeled exercise metric results from the different user category to determine the one or more features. The labeled exercise metric results from the different user category may be used to determine an initial set of features, which may be further reduced using the training data set.

The training method may train one or more machine learning models using the one or more features. In one embodiment, the machine learning models may be trained using supervised learning. In another embodiment, other machine learning techniques may be employed, including unsupervised learning and semi-supervised. The trained machine learning models may be selected based on different criteria depending on the problem to be solved and/or data available in the training data set. For example, machine learning classifiers can suffer from different degrees of bias. Accordingly, more than one machine learning model can be trained and then optimized, improved, and cross-validated at a subsequent step.

The training method may select one or more machine learning models to build a predictive model (e.g., the at least one machine learning-based classifier). The predictive model may be evaluated using the testing data set. The predictive model may analyze the testing data set and generate classification values and/or predicted values. Classification and/or prediction values may be evaluated to determine whether such values have achieved a desired accuracy level.

Performance of the predictive model described herein may be evaluated in a number of ways based on a number of true positives, false positives, true negatives, and/or false negatives classifications of exercise metrics of users. For example, the false positives of the predictive model may refer to a number of times the predictive model incorrectly classified a exercise metric(s) as indicating a particular attribute that in reality did not indicate the particular attribute. Conversely, the false negatives of the machine learning model(s) may refer to a number of times the predictive model classified one or more exercise metrics as not indicating a particular attribute when, in fact, the one or more exercise metrics did indicate the particular attribute. True negatives and true positives may refer to a number of times the predictive model correctly classified one or more exercise metrics of a user as having sufficient indication of a particular attribute or not indicating the particular attribute. Related to these measurements are the concepts of recall and precision. Generally, recall refers to a ratio of true positives to a sum of true positives and false negatives, which quantifies a sensitivity of the predictive model. Similarly, precision refers to a ratio of true positives to a sum of true positives and false positives. Further, the predictive model may be evaluated based on a level of mean error and a level of mean percentage error. Once a desired accuracy level of the predictive model is reached, the training phase ends and the predictive model may be output. However, when the desired accuracy level is not reached a subsequent iteration of the method may be performed with variations such as, for example, considering a larger collection of exercise metrics of historical users.

### Exemplary Vehicle with Propulsion Assistance

### Introduction

A vehicle having propulsion assistance is disclosed. The vehicle can be configured for transportation and/or for use as an exercise apparatus. The vehicle can be, for example, a wheelchair or a cycling device (e.g., a recumbent tricycle). It is contemplated that the user can have sufficient strength to propel the vehicle on a smooth, flat, or declined surface. However, the user may not have sufficient strength to traverse an upward incline or a rough surface. In these aspects, the vehicle can comprise an orientation sensor that can be configured to determine a slope on which the cycling device is traveling. The orientation sensor can further be used to determine a terrain condition (e.g., roughness). The orientation sensor can be in communication with a controller that is configured to control a power output of a motor that is operatively coupled to a wheel of the cycling device. Similar features can be applied to other vehicles, such as, for example, a wheelchair.

### Exemplary Vehicle with Propulsion Assistance

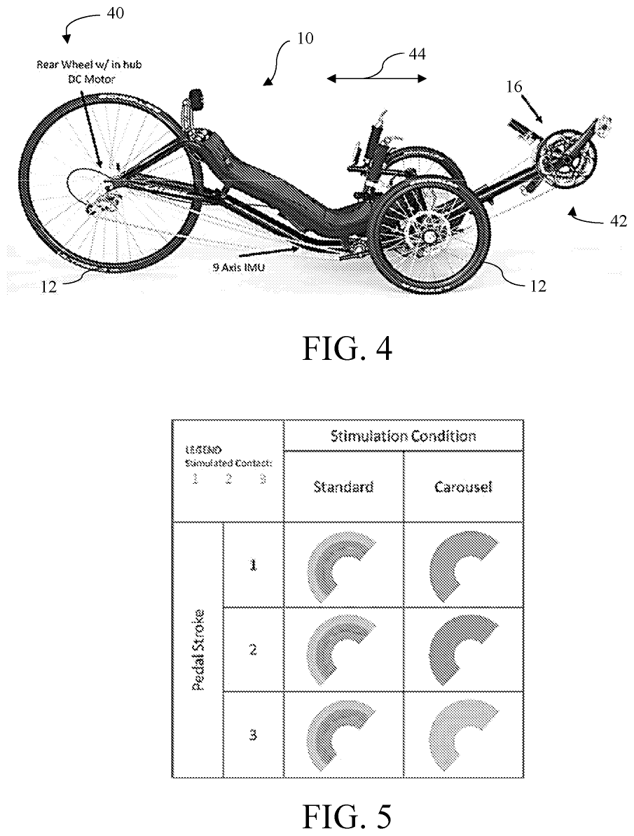

Disclosed herein, and with reference to FIGS. 4, 15A, and 15B is a vehicle 10 that is movable along a surface. The vehicle 10 can comprise a plurality of wheels 12. The vehicle can be configured to be at least partly powered by a user. For example, the vehicle 10 can be a cycling device that the user can pedal via a crankset. In other aspects, the vehicle 10 can be a wheelchair that can receive user power via a pushrim.

The vehicle 10 can comprise a propulsion assist system 20. The propulsion assist system 20 can be configured to supplement the power that the user inputs via the crankshaft 16. The propulsion assist system 20 can have at least one battery 22 and a motor 24 that is operatively coupled to at least one wheel of the plurality of wheels and configured to cause rotation of the at least one wheel of the plurality of wheels. The motor 22 can be a brushed motor or a brushless motor.

A controller 30 can be in electrical communication with the motor 24. At least one orientation sensor 32 can be in communication with the controller 30. The at least one orientation sensor 32 can be configured to determine a sensed orientation of the vehicle 10. For example, the at least one orientation sensor 32 can be configured to determine whether the vehicle is on an uphill slope, a downhill slope, or on flat ground. In some aspects, the at least one orientation sensor 32 can comprise an inertial measurement unit. In some aspects, the at least one orientation sensor 32 can comprise 3-axis accelerometer, a 3-axis gyroscope, and a 3-axis magnetometer. In some aspects, the vehicle 10 can have a front portion 40, a rear portion 42, and a longitudinal axis 44 that extends between the front portion and the rear portion of the vehicle. The sensed orientation can comprise an orientation of the longitudinal axis of the vehicle relative to a horizontal plane (e.g., an uphill orientation or a downhill orientation).

The controller 30 can be configured to modulate a power output of the motor based at least in part on the sensed orientation of the vehicle 10. For example, the controller 30 can increase output from the motor 24 when the vehicle 10 is traveling uphill and decrease the output from the motor (or apply a resistance) when the vehicle 10 is traveling downhill. In some aspects, optionally, the controller 30 can determine the torque for the motor to apply to offset an incline (so that the motor delivers the additional torque/power so that the combined output from the user and the motor is equivalent to the power that the user needs to provide on flat ground to maintain speed). Optionally, the controller 30 can cause the motor 24 to apply zero torque on flat ground. In other aspects, the controller 30 can cause the motor 24 to apply a first torque when on flat ground and a second torque that is greater than the first torque when the vehicle is traveling up an incline.

In further aspects, the controller 30 can be further configured to, based on feedback from the at least one orientation sensor 32, determine a terrain condition. For example, the controller 30 can perform a fast Fourier transform (FFT) on the feedback from the at least one orientation sensor 32 to determine a terrain condition. The controller 30 can configured to modulate a power output of the motor 24 at least in part based on the terrain condition. For example, the terrain condition can be a roughness. The controller can be configured to increase the power output of the motor over rough terrain and decrease the power output of the motor over smooth terrain.

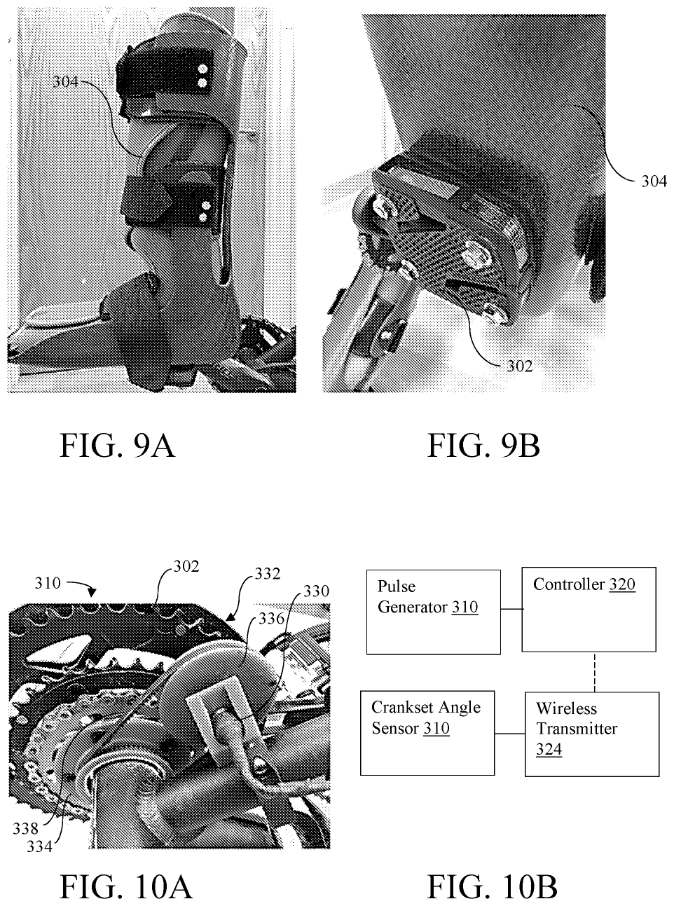

The vehicle 10 can be a cycling device, such as, for example, a recumbent tricycle. The cycling device can comprise a crankset 16 for receiving power from a user. The crankset 16 can be in communication with at least one wheel of the plurality of wheels 12. The cycling device can further comprise a pair of appendage receptacles (e.g., boots) that are coupled to the crankset. Optionally, as illustrated in FIG. 9A, the pair of appendage receptacles can each be configured to immobilize a respective joint (e.g., ankle) of the user.

In some aspects, the motor 24 can be configured to apply a torque to the at least one wheel in a rotational direction that corresponds to forward movement of the vehicle. In other aspects, the motor can be configured to apply a torque to the at least one wheel in a rotational direction that resists forward movement of the vehicle.

In some optional aspects, the controller 30 can be configured to determine a position of the crankset based on feedback from the at least one orientation sensor. In other aspects, the vehicle can comprise a crankset position sensor that is configured to determine the position of the crankset.

In some optional aspects, the vehicle 10 can comprise a functional neural stimulation system as disclosed herein. For example, the vehicle 10 can incorporate one or more aspects disclosed herein under the heading “Exercise Apparatus with Functional Neural Stimulation” or in any of the following examples. Thus, in some aspects, the vehicle 10 (FIG. 4) can be the exercise apparatus 300 (FIG. 8). In other aspects, the vehicle 10 does not comprise a functional neural stimulation system.

A method of using the disclosed vehicle can comprise sensing, by at least one orientation sensor, an incline of the vehicle. A power output of the motor can be controlled based at least in part on the incline of the vehicle.

The method can further comprise sensing, by the at least one orientation sensor, a terrain condition upon which the vehicle is traveling. The power output of the controller can be controlled based at least in part on the terrain condition.

### Computing Device

FIG. 28 shows an operating environment 1000 including an exemplary configuration of a computing device 1001 for use with the system 200 (FIG. 8). Optionally, all or portions of the computing device 1001 can be integral to the exercise apparatus 300. Further, the controllers 30 and 320 can optionally be embodied in accordance with the description of the computing device 1001 and/or remote computing device 1014a,b,c. Optionally, the computing device 1001 can comprise a tablet, a computer, a smartphone, or other suitable structure.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as parameter data 1007 and/or program modules such as operating system 1005 and parameter setting software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and parameter setting software 1006 may be stored on the mass storage device 1004. One or more of the operating system 1005 and parameter setting software 1006 (or some combination thereof) may comprise program modules and the parameter setting software 1006. The parameter data 1007 may also be stored on the mass storage device 1004. The parameter data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Exemplary Embodiments and Test Results

### Example 1

Various aspects disclosed herein are directed to stimulation-induced exercise for individuals with paralysis. With participants seated on specially adapted exercise equipment, such as recumbent tricycles with orthotic boots affixed to the pedals or adapted rowing machines with back support for stability, custom stimulation models can deliver spatial and temporal patterns of electrical pulses through surface and/or implanted electrodes that produce coordinated movements in the otherwise paralyzed limbs. Stimulation patterns can utilize sensors, such as angle encoders, inertial measurement units, or linear potentiometers to determine instantaneous positions of the extremities and determine which muscles should be activated at a given point in time and at what intensity to effectively complete the a movement (e.g., a pedal stroke or rowing maneuver). Existing systems use non-adaptive stimulus levels and timing schemes, induce rapid muscular fatigue, and do not engage the participants or consider their instantaneous physiological state.

Improvements upon existing stimulation-induced exercise systems are multi-faceted. First, a carousel stimulation pattern is provided, in which synergistic muscle fiber groups are activated sequentially (instead of simultaneously) during appropriate portions of the exercise movement. For example, a single group of knee extensor fibers can be activated one cycling pedal stroke, while a separate group of knee extensor fibers may be activated the next pedal stroke, as shown in FIG. 5. This allows some fibers to briefly rest and recover while others maintain the desired movement in order to reduce fatigue. Stimulation model logic behind carousel control schemes uses feedback from the exercise apparatus to keep track of cycling pedal stroke or rowing repetition number to activate the desired subset of muscle fibers each time. For example, in a carousel cycling scheme, each time a pedal stroke passes a reference crank angle outside of a muscle's activation angles, the model updates which a subset of fibers are activated during the subsequent pedal stroke, as shown in FIG. 6. Carousel stimulation schemes can include at least two (optionally, three, four, or more) independently controlled electrode contacts that activate separate yet synergistic motor unit pools in order to rotate between them without overlap, and may include as many fiber pools as is available and desired. Timing schemes for rotation of stimulation can be once per cycle as described, or may be more rapid, with multiple fiber groups being sequentially activated within each cycle. This example shows use of a carousel pattern in which one knee extensor group is activated each pedal stroke during stimulation-induced cycling.

In addition to carousel stimulation, disclosed in this example is a closed-loop control scheme to adjust stimulus levels (e.g., one or more of: pulse duration, amplitude, or frequency) throughout an exercise based on a desired target output (speed/cadence or work performed). Closed-loop control schemes can use feedback from the exercise apparatus, such as the rate of change of the cycling crank angle encoder as an approximation of cycling cadence, to adjust stimulus level, as illustrated in FIG. 2. Conventional cadence sensors calculate cadence once per revolution (i.e., how long it takes to complete a full revolution from 0 to 360 degrees) and report that cadence only once per second. By using the instantaneous derivative of the change in crank angle from the encoder signal, a much higher resolution signal for feedback control can be obtained, thereby allowing for finer adjustments to the stimulation intensity throughout each pedal stroke. Using a derivative of a signal that repeatedly cycles from 0 to 360 degrees and back requires special handling of the 360-to-0 transition point, where the derivative would become negative despite cycling still progressing in the forward direction. This can be accounted for with special logic within the controller that stores the last positive value of the cadence until that transition phase is over. It can be shown that modulating stimulus levels with proportional-integral or proportional-integral-derivative (PID) control systems to match a submaximal but higher than steady-state (e.g., fatigued) exercise intensity using this model improves endurance and enables participants to cycle longer, often accumulating more work done prior to fatigue than with conventional stimulation. Setting the submaximal target cadence can be done by a clinician or the user at the beginning of an exercise session, or interactively throughout the session with a throttle-like input device to allow the user full command of their pedaling speed on a trainer or overground.

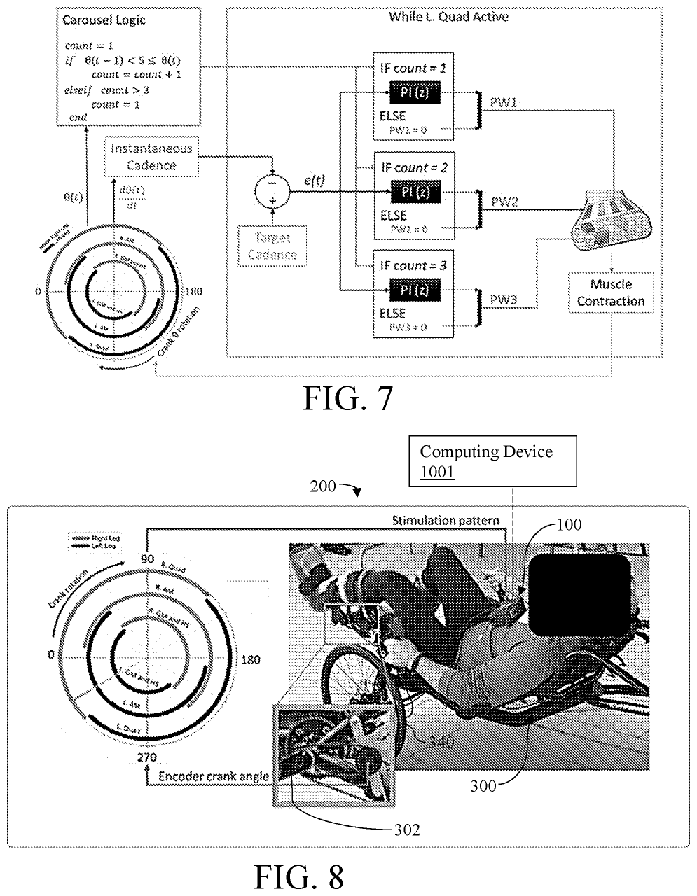

The closed-loop control schemes can be extended to automatically adjust stimulation pattern timings (i.e., the crank ankles/rowing positions that stimulation turns on/off) based on the same feedback signals mentioned above to advance or retard stimulation according to speed or cycling/rowing cadence. This is advantageous because static and unchanging stimulation on/off times can be optimal when exercise occurs at one specific rate due to the delays and dynamics of the contractile properties of the muscles. When the rate of exercise slows down, activation of some muscles can be delayed to prevent antagonists from fighting each other, while when exercise rates increase, some muscles can receive activating stimuli sooner to ensure they are fully contracted by the time they are needed. Iterative or reinforcement learning control (ILC or RLC) and/or dynamic analyses of the system (or other machine learning techniques including artificial neural networks) can be employed to achieve this adaptive behavior. Feedback controlled stimulation schemes can be used on their own or in combination with carousel patterns from above, as shown in FIG. 7. An error e (t) between target and instantaneous cadence can drive a PI controller to adjust the PW delivered through all knee extensor-activating contacts during the left and right quadriceps' respective active periods of the pedal stroke.

A third aspect of this examples is the incorporation of virtual/augmented reality (VR/AR) and other methods of engaging the user/monitoring their volitional effort during the exercise. Because stimulation is driving the movements of paralyzed muscles in these exercise systems, there is no volitional control and participants often become disengaged from the exercise. Consciously re-engaging the user is important because studies have shown central drive, or the command from the brain to muscles telling them to move, is key to initiating higher heart rates, blood pressures, and other physiologic responses during exercise that are important for effective cardiovascular functioning during exercise. The absence of this central drive, in addition to other limiting factors of paralysis, cause physiological responses to remain barely elevated from resting values, which can prevent the activated muscles from being sufficiently supported for continued work, limit endurance, and compromise the intensity of exercise that is required for reconditioning. Finding ways to refocus a participant on the exercise can thus prove extremely valuable to both their functional performance and their investment in continuing an exercise regimen.

Additionally or alternatively, a way to engage the users is to provide real-time feedback of their volitional effort during the exercise. Effort in muscles they still have control over, such as their upper extremities, can influence heart rate and other responses, so encouraging them to concentrate on and perform work in those muscles while cycling or rowing may help supply the lower extremities while also getting a better full body workout. For example, hand grasp force measurement devices can be incorporated into the bike or rowing machine handles and their outputs displayed on a screen to encourage participants to squeeze hard or along with different patterns as a way to increase heart rate and blood pressure. Ventilation measures such as respiration rate and volume can be integrated into the VR headsets and displayed to encourage deeper breaths at a desired pace, as normal pulmonary responses can also be blunted after paralysis. This particular feedback signal would encourage better oxygen supply as well as necessitating conscious focus and engage the central drive. Additionally, recent research shows that even in people whose spinal cord injury is classified clinically as motor complete, small EMG signals may be detected from muscles below the level of injury, even if they are not strong enough to produce a functional movement in those muscles. These spared EMG signals could also be used as a feedback signal as a measure of volitional effort.

These and other physiologically-based signals can be incorporated into VR games as feedback signals for encouragement and/or a control signal for the stimulation itself to match stimulated exercise intensity with current physiological capacity (think: treadmill that automatically adjusts speed based on heart rate to keep you in a target zone, but this would adjust stimulation) or effort level (they may only move in the VR scene if sufficient effort via a physiological signal was detected). These VR/AR approaches are being integrated with the open and closed loop control paradigms for a complete system to facilitate more effective exercise after paralysis.

Another aspect of VR/AR disclosed is a communal video platform that allows individuals with disabilities to ride together virtually in simulated cycling outings or races, regardless of their physical or geographic locations. This “Stimulation-Powered Cycling Community” can utilize the commercially available ZWIFT platform with custom modifications to ensure that users of different ability levels, power output and duration can ride together regardless of their individual capabilities, with the speed of their respective avatars appropriately scaled to ensure that the participants remain closely grouped so they can interact effectively. The engagement with the community and recreational and social aspects of cycling together can be motivating and can encourage users to exercise regularly in a way that is more effective than riding alone.

### Exemplary Aspects Include

A system and method to alternate between synergistic muscles during exercise driven by neural activation after paralysis to prolong joint moment and total power output, enabling more intense exercise for longer durations when stationary or extended distances over ground compared to conventional stimulation.

A system and method to modulate stimulus parameter (pulse duration, amplitude and/or frequency) to maintain a desired sub-maximal cadence or power output for long durations above the fatigued steady state, thus enabling more intense exercise for longer durations or distances. This innovation also lends itself to cadence (speed) controller that allows the user to adjust their speed with a throttle or some other input device.

A control system that is self-tuning via Machine Learning (AI) methods and can adapt to various speeds to advance or retard stimulus timing to coincide with optimal muscle contractions and compensate for intrinsic delays and dynamics of paralyzed muscles activated by neural stimulation.

Virtual or Augmented Reality (AR/VR) systems to re-engage the central drive and improve the intensity of workouts by normalizing hemodynamic and pulmonary responses to exercise of the large lower limb muscles.

A method for establishing, organizing, and conducting a “Stimulation-Powered Cycling Community” that permits pilots to ride together and interact virtually in recreational outings or competitions and displays avatars appropriately scaled to the capabilities of each rider to ensure effective interactions regardless of individual capabilities.

A combination of all of the above for reconditioning training, health maintenance, reversal of secondary clinical complications such as atrophy, bone density loss, and cardiac deconditioning, as well as recreational use over ground over significant distances and durations.

### Example 2

Adaptive cycling for individuals with spinal cord injury, stroke, and other disabilities has been performed with a cycling device (a recumbent tricycle) equipped with ankle/foot immobilizing orthotics and a crank position sensor and a functional electrical stimulation (FES) (also referred to as functional neural stimulation (FNS)).

Exemplary aspects include gearing to measure crank angle, foot-pedal attachment to immobilize the ankle and control hip ab/adduction, a wireless transmission system, an external control unit capable of providing electrical stimulation via radio frequency to implanted pulse generators, percutaneous electrode leads, or surface electrode pads, and a clinical interface app. Other aspects of this disclosure include one or more wireless inertial measurement unit(s) (IMU) placed on the crank arm(s) to measure crank position or added electromechanical assist to compensate for fatigue or difficult terrains. An exemplary embodiment comprises a recumbent tricycle with custom developed components to enable individuals with paralyzed muscles the ability to ride stationary via a commercial trainer or overground in community settings.

Referring to FIGS. 9A and 9B, to immobilize the ankle, which can be advantageous to control the hip ab/adduction due the paralyzed musculature, a foot-pedal attachment mounting is disclosed. A generalized carbon fiber ankle foot orthosis (AFO) can be molded to fit a variety of leg sizes and can be filled with padding to ensure good fit and proper skin protection. The AFO for biking can secure the user at the midfoot, above the ankle, and below the knee to constrain the ankle and keep the thighs (hip and knee joints) aligned and protected from injury. A flat pancake pedal can be mounted directly under the heel to optimize transmission of force directly from the tibia via fasteners (e.g., bolts) to the bottom of the reinforced carbon fiber orthosis. A heel portion of the AFO can be constructed to have a flat interface to fit flush with the pedal.

Referring to FIGS. 10A and 10B, to measure crank angle, which can be advantageous to know accurately to precisely time the muscle activation, a gearing with rotary encoder was developed and is mounted to the crankset. A toothed pulley (which can optionally be 3D-printed) can be bolted to the crankset and can be connected via a belt to another toothed pulley that is coupled to a rotary encoder. This enables the rotary encoder to rotate proportionately to (optionally at a 1:1 ratio) the crank to accurately measure the crank location so the system knows the location of the legs at all times. The rotary encoder output is routed to a transmitter (optionally, a wireless transmitter) that can communicate with the rest of the system (e.g., via a 900 MHz radio frequency link).

Referring to FIGS. 11A and 11B, another embodiment of this disclosure can be configured to determine crank angle using an IMU on one or both of the cranks, similar to how commercial cadence sensors mount. The IMU(s) can be a 9-axis sensor comprising a 3-axis accelerometer, 3-axis gyro, and 3-axis magnetometer. The sensor output signals can be fused through a Kalman filter to produce orientation of the cranks. This orientation can be wirelessly transmitted to a controller to determine crank position and stimulate the muscles at the appropriate time. A single IMU on a crank can produce the crank angle. In further aspects, multiple IMUs can be used in tandem to also determine the incline of the bike to account for hills and adjust the stimulation accordingly.

Referring to FIG. 12, the wireless transmitter communicates with a controller (e.g., an Application Specific Control Unit (ASCU)) that can serve as the main hub for communication and stimulation output. In exemplary, optional aspects, the controller can comprise a TEENSY embedded control board, BLUETOOTH module, 900 MHz receiver board, a custom power/interface board, and stimulation boards. The controller can be housed in a 3D printed enclosure with a lithium-ion rechargeable battery. Stimulation boards can provide stimulation via RF coupled inductive links with implanted devices, percutaneous leads, or via surface stimulation. The ASCU can receive the crank position (e.g., via the wireless transmitter) and maps the crank angle to stimulation output. As the crank angle changes via the sensor, the stimulation output changes. This can provide improved pedaling, including smooth pedaling rotation.

To control and program the stimulation patterns, a clinician-friendly tablet interface app can be used. For example, a tablet can run an Android-based app that communicates wirelessly with the stimulator Bluetooth module. An exemplary screenshot of this tablet-based application is shown in FIG. 13. This application allows the clinician to enter stimulation parameters (e.g., amplitude, pulse width, stimulation on-crank angle, and stimulation-off crank angle) for each stimulation channels as well as well the stimulation interpulse interval. A circular graph is shown that automatically updates as stimulation parameters are changed. This gives the clinician a visual representation of the timing of all stimulation channels. The icons located at the bottom of the app allow the clinician to start/stop cycling, calibrate the encoder, test individual stimulation channels, and send the stimulation parameters to the external control unit over the Bluetooth link. A separate page of the application allows for customizable training regimens to be programmed and automatically run.

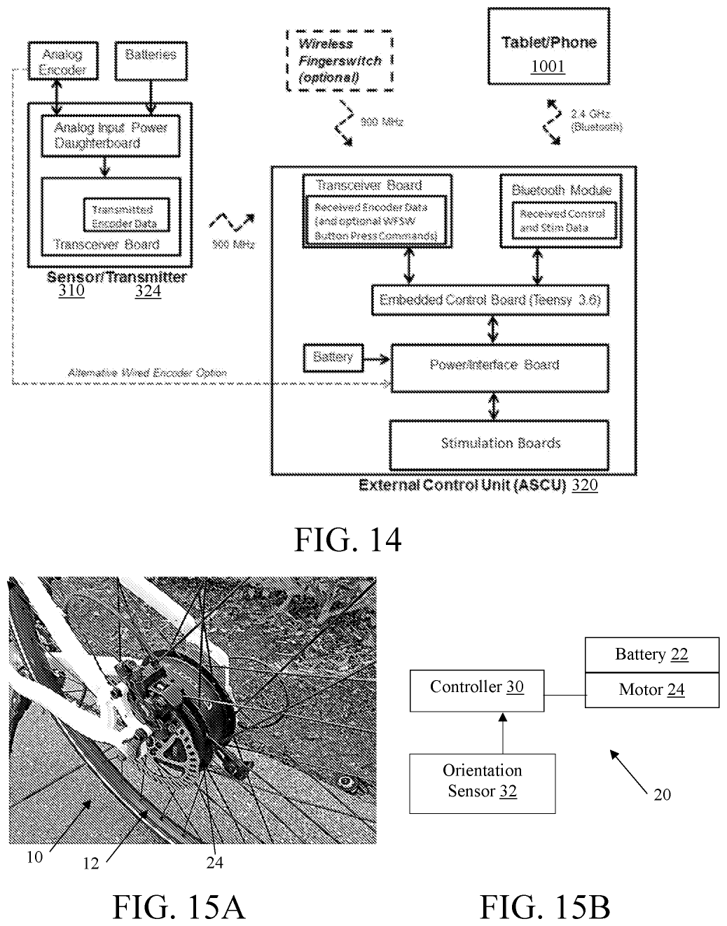

Advantages of the developed system described here is that is possible to use it stationary via a commercial cycling trainer or overground, which is not possible with many electrical stimulation systems available today. It also is versatile in that it can be used with multiple stimulation methods (implanted, percutaneous, or surface). A block diagram of an exemplary system is shown in FIG. 14.

Other embodiments of this system can comprise alternative power sources to assist the electrical stimulation for long journeys or up inclines. For example, the cycling device can comprise an on-demand electromechanical assist system designed to assist the user in instances of fatigue or cases where high power is needed (i.e. inclines). This can be accomplished via an electric motor with a controller integrated into the ASCU that automatically determines when assistance is required. The data can be fed through a feed-forward model that can predict the incremental torque needed to navigate the terrain, allowing the stimulation or user supplied volitional torque to still provide most of the propulsive torque. In effect, whenever the user rides uphill or over rough terrain, this system can cause the user to experience riding over flat, level ground.

### Example 3

A recumbent tricycle (equipped with ankle/foot immobilizing orthotics and a crank position sensor) and a functional electrical stimulation (FES) has been used with individuals having spinal cord injury. Implanted stimulation systems that connect an external control unit (ECU) via percutaneous wires or an RF coupled inductive coil have been used. In some aspects, commonly utilized surface electrodes can be used.

Lab testing has shown that stimulation users are typically unable to produce peak or steady state power similar to what a typical able body user is capable of. In most cases, steady state power levels range between 20-40 watts. From a physical perspective, this level of power is adequate to propel the user over a relatively slick surface with no hills or terrain features. Experience has shown that when confronted with a rough surface (like a rubber track) or a slight incline, the user struggles to maintain forward progress, and typically must use her arms to supplement the stimulation and drive her legs.

Disclosed herein is an on demand electromechanical assist system configured to provide a layer of safety and reassurance to the user. The conventional motorized assist system relies on a manual thumb throttle, with the user manually indicating when they would like assistance. The disclosed system comprises sensors to automatically determine when assistance is required (such as hill or rough terrain). This approach enables maximization of the stimulation driven effort of the user and only supplements power when the system determines additional power/torque is required. The disclosed system can be effective with individuals having spinal cord injury or any disease/injury that results in reduced strength or weakened lower extremity muscles.

The system can comprise a motor, controller, battery, and sensors. The motor, controller, and battery form the power unit-battery for energy storage, motor to provide propulsive power, and a controller to act as a throttle for the system. At least one sensor can be an orientation sensor (e.g., a 9-axis Inertial Measurement Unit (IMU)). A 9-axis IMU can comprise a 3 axis accelerometer, 3 axis gyro, and 3 axis magnetometer. These sensor signals can be fused through a Kalman filter to produce roll/pitch/yaw Euler angles. This application can primarily be concerned with the pitch orientation angle—this can indicate whether the user is climbing a hill. Additionally, the vertical acceleration can help determine a terrain condition (e.g., the roughness of the terrain being ridden)—by running a Fast Fourier Transform (FFT) on the vertical acceleration signal. Rougher terrain can have a more active frequency spectrum than flat terrain. Consequently, rougher terrain can require more power to navigate.

An electric motor can be used to provide the assistive torque to the system. The motor can be embodied in various ways—the motor itself can be either brush or brushless, operating of DC voltage. A transmission can be used to multiply the torque of the motor to a level appropriate for bike propulsion. This motor can be positioned in a variety of locations, including at the crank, embedded in the hub of the rear wheel (as illustrated in FIG. 15), or someplace else.

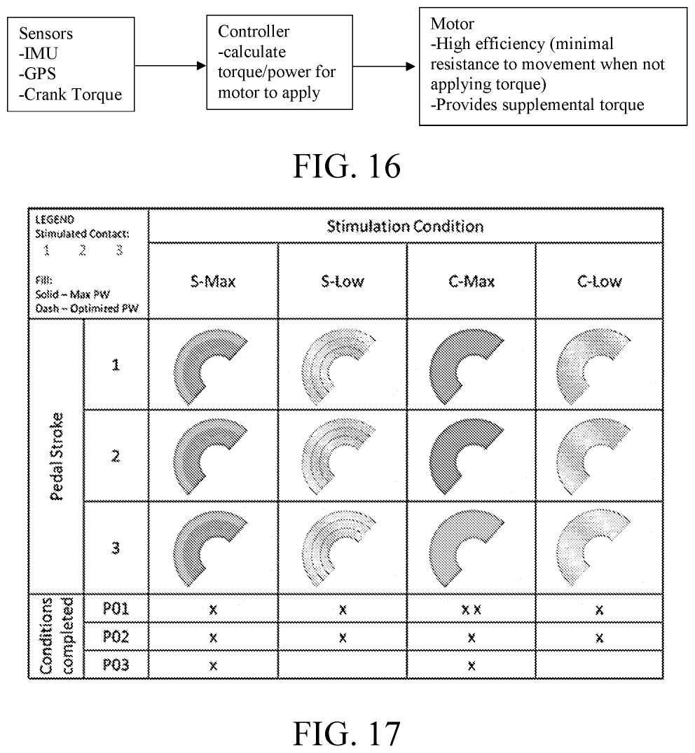

Referring to FIG. 16, sensor data can be fed through a feed-forward model that can predict the incremental torque needed to navigate the terrain, allowing the stimulation or user supplied volitional torque to still provide most of the propulsive torque. In effect, whenever the user rides uphill or over rough terrain, the system can adjust the effort required so that the user still experiences riding over flat, level ground. This system can provide confidence and redundancy to the stimulation-allowing for more adventurous, out of lab excursions in a less controlled environment. An advantageous feature of this approach in this application is that the control algorithm can be specifically designed to maximize the effort of the rider before applying assistive torque. When applied to biking as a form of cardiovascular exercise, this is critical to obtaining maximum health benefit. Other motorized cycling applications may continuously run their motor to ensure a constant speed, however this approach can minimize the effort exerted by the rider. While embodiments of the present disclosure can be embodied in adaptive cycling, the concept of context dependent robotic assistance is also applicable to other situations. An electromechanical exoskeleton can operate under the same principle-allowing the user to walk primarily using their implanted stimulation system, while sensors monitor the motion of their legs. If the onboard intelligence detects that stimulation alone is unable to complete the step, it is able to trigger electromechanical actuators located at the user's joints, supplementing the biological torque with an incremental torque to complete the motion.

Another embodiment can be used in wheelchair propulsion. For example, a high level thoracic or cervical injury may result in upper extremity weakness. While certain users can still possess the strength to push themselves in a manual wheelchair over level ground, uphill terrain may be a challenge. A system can comprise: one or more sensors that detect uphill or rough terrain, and a motor that supplies an incremental torque to supplement the user's contribution. This offers advantages over commercially available power-assist wheelchairs with motorized hubs, which lack any ability to sense the challenges of difficult terrain and adapt their level of motor assistance to the situation. In those devices, the force applied to the push rim is amplified the same way every stroke, regardless of whether the user is attempting to ascend or descend inclines. The key difference in the disclosed system is that the system can be passive (seemingly invisible to the user) when assistance is not needed, and only provide assistance when needed. This can provide a large boost in effective run time and battery life vs. a system that is always active.

The electromechanical actuator can also be used to apply a resistive torque, as well as, or as an alternative to, a supplemental torque. This can also be useful in certain scenarios. For example, in the case of biking or propelling a manual wheelchair downhill, the motor can act as a brake, ensuring that speeds remain in a safe range. In this case, the IMU/orientation sensor can detect downhill inclinations and apply a resistive torque based on a computed feed forward model.

This aspect can also be used for resistive training of cycling. Experience has shown that commercial stationary trainers that use magnets or fluid to provide a resistive load to the user typically lack resolution in the power range of interest of the stimulation enabled rider. Even the lowest resistance setting can be too much for the reduced power output of stimulation enabled riding. A properly sized actuator can provide appropriate levels of resistance for effective training.

Exemplary aspects herein can be embodied as a context dependent robotic assistance system. It can also be applied to rehabilitation and assistive robotics, including wearable robots for walking or gait training after paralysis or stroke (i.e., exoskeletons). In those devices, the internal friction and passive resistance need to be overcome by the active contractions of weak or paretic muscles. Compensating for the resistance of the mechanism itself can allow users to be more efficient at moving the device and better able to walk or engage in rehabilitation training activities. In some aspect, an exemplary system can comprise an electromechanical actuator, sensors to interact with the physical world, and computational intelligence to determine when intervention is necessary, as well as how much intervention is necessary. The intent of the exemplary system is to not operate autonomously, but to interact with a human user, and supplement what that user is already providing. Presented are multiple embodiments of this concept within the scope of APT center research, but undoubtedly there are many other embodiments in other areas of use. In this context, this control strategy can obtain both advantages previously discussed-on demand assistance can maximize the effort of the user (with maximum health benefits, same as the biking application), while also maximizing battery life and range compared to alternative solutions.

### Example 4

One potential way to acutely improve electrically-induced exercise is to reduce the overlap of activated fibers among stimulating electrodes. Current systems stimulate through multiple surface electrode pads or implanted neural electrode contacts at once and at high pulse amplitudes (PA) and/or pulse widths (PW) to engage as many muscle fibers as possible, particularly during knee extension phases of cycling. Though this can result in high initial power production, the large voltage fields produced by each electrode can overlap and limit performance as the exercise goes on. Stimulation through multiple electrodes is rarely perfectly synchronized, so motor units within the overlapping regions can be forced to fire at higher frequencies than intended due to the summation of the slightly asynchronous fields. For example, a motor unit within a region of overlapping fields from two electrodes stimulating individually at 20 Hz can experience a combined firing frequency demand of 40 Hz. Higher firing frequencies have been shown to increase rates of fatigue, so these overlapping fields likely contribute to the considerable decline in force and power production seen shortly after the onset of stimulation. Using selective, multi-contact nerve cuff electrodes and overlap optimization techniques described in, stimulation levels can be adjusted through individual electrode contacts to provide ample muscle recruitment with minimal field overlap, which may improve cycling performance.

A second approach that may acutely improve stimulation-driven exercise is to reduce the duty cycle of activated motor units. Conventional cycling stimulation methods activate large groups of synergistic muscle fibers each pedal rotation. For example, large portions or even multiple heads of the quadriceps are activated concurrently when strong knee extension is needed. The activated fibers thus have a high duty cycle, or work to rest ratio, as they are all repeatedly activated each pedal stroke. Studies have shown that high duty cycles contribute to rapid muscle fatigue and force decline, whereas lower duty cycles can extend muscle output prior to fatigue. Duty cycle may be lowered without interrupting cycling motion by alternating between muscles with a “carousel” stimulation pattern through selective, multi-contact electrodes. Carousel stimulation rotates activation among multiple independent yet synergistic subsets of fibers such that one performs the desired action while the others rest and recover. By activating only one independent motor unit pool (MUP) each pedal stroke and alternating which pool is active or resting every pedal rotation, duty cycle can be decreased and cycling performance improved.

The goal of this study is to explore the relative effects of low overlap stimulation and low duty cycle stimulation in isolation and in combination to determine their acute effects on cycling performance after SCI. It is contemplated that reducing the overlap and/or duty cycle of activated fiber groups can increase functional work performed within an exercise session over conventional stimulation techniques.

Three individuals with SCI with implanted neural stimulation systems (FIGS. 1A-1F) customized for other studies of standing, stepping or transfers in the laboratory participated in the selective stimulation-driven exercise experiments. A crank angle encoder (US Digital, Inc.) relayed instantaneous recumbent bike pedal crank position to an external control unit (ECU) running custom cycling exercise stimulation models as a Sim-ulink real-time xPC target. Crank angle was mapped to the necessary muscle activations and timings for smooth cycling within the ECU stimulation model. The ECU relayed the desired stimulus based on crank angle via a close coupled inductive radiofrequency communications link to a subcutaneous implanted pulse generator. The implanted stimulator then delivered appropriate charge balanced, current controlled, asymmetric, pulse width modulated waveforms through intramuscular or epimy-sial electrodes near the motor nerves of the desired hip and trunk muscles, or through individual multi-contact nerve cuff electrode contacts on the femoral nerves to activate individual portions of the quadriceps group (FIG. 2). The implanted components of this system have been shown to provide stable longitudinal performance without damage to the stimulated neural tissue.

The quadriceps, hamstrings, adductors, and gluteal muscles may all be involved in the stimulation patterns to generate cycling exercise. For this study, only activation of the quadriceps (knee extensors) varied among stimulation conditions. In two participants (P02 & P03), composite flat interface nerve electrodes (C-FINEs) were bilaterally implanted around the proximal femoral nerves. Prior studies found three contacts on each C-FINE could selectively activate independent knee extensor MUPs in such cases. The other participant (P01) had bilateral spiral cuff electrodes around the proximal femoral nerves and epimysial electrodes sutured near the motor point of each vastus lateralis (VL). Two contacts per spiral cuff and the epimysial electrode were found to selectively activate independent knee extensors. Each participant therefore had three independently controlled electrical contacts that elicited separate MUPs for knee extension, enabling the study of the effects of overlap and duty cycle. A summary of participant demographics and implanted electrodes of interest to this study is provided in Table 1.

TABLE 1

| | | | Knee Extensor Stimulation |
|---|---|---|---|
| Participant | SCI Classification | Activity Level (rides per week) | (Independent Contacts) |
| P01 | C7 ASIA-B | High (3-4) | Proximal femoral spiral cuffs (2) |
| | | | VL epimysial (1) |
| P02 | T10 ASIA-A | Moderate (1-2) | Proximal femoral C-FINEs (3) |
| P03 | C7 ASIA-C | Low (<1) | Proximal femoral C-FINEs (3) |

### Cycling Protocol and Stimulation Conditions

While seated in a recumbent tricycle (Catrike, Orlando, FL) with their legs secured in custom orthotic boots secured to the pedals, participants used their arms to manually cycle their legs for approximately one minute to overcome initial tightness and spasticity in the paralyzed musculature. If necessary, additional stretches were performed until manual cycling was easily achieved without excessive resistance from underlying muscle tone. Participants then performed stimulation-induced cycling trials with up to five different stimulation conditions to determine effects of fiber overlap and duty cycle on exercise outcomes (FIG. 17). FIG. 17 illustrates cycling exercise stimulation conditions completed by each participant. Colors represent the electrical contact(s) delivering stimulating current through each pedal stroke for a single leg and fills represent stimulation level. (1) S-Max is the conventional pattern which activates multiple MUPs each pedal stroke at supramaximal intensities. (2) S-Low activates multiple MUPs each pedal stroke at optimal PWs found through moment summation tests to reduce overlap among activated fibers. (3) C-Max activates one MUP per pedal stroke at supramaximal intensities, rotating active MUP each revolution to reduce duty cycle. (4) C-Low combines low duty cycle and low overlap approaches by rotating activation of a single MUP each pedal stroke and stimulating at optimized PW levels. Note that P01 completed two variations of the C-Max stimulation condition involving different numbers of independent MUPs.

The Standard, Maximum Overlap (S-Max) condition represents conventional stimulation. Each knee extensor MUP is supra-maximally activated (Frequency=25 Hz, PA=0.8 mA, PW=255 μs) each pedal rotation, resulting in both high duty cycles and high overlaps among activated MUPs. The Standard, Low Overlap (S-Low) condition similarly activates each knee extensor MUP every pedal rotation, but uses optimized stimulation PW values found through moment summation tests on a dynamometer prior to exercise to reduce overlap among activated fibers. Moment summation tests examined the difference between actual moment output when one MUP is stimulated within the refractory period of another, and ideal summation of the outputs generated when each MUP was stimulated individually. Perfect summation indicated completely independent yet synergistic MUPs. A difference between actual and ideal summation was used to calculate functional percent overlap. Moment summation tests were performed across a wide range of PWs through each involved contact to identify those that generated enough muscle output for a given task while keeping overlap below a chosen threshold. See reference for more details. These optimization procedures generally resulted in submaximal stimulus levels delivered through each involved contact in the S-Low condition.

A summary of the optimized stimulation levels and corresponding decreases in overlap compared with conventional stimulation is provided in Table 2. P03 did not complete overlap testing sessions due to time limitations and upper extremity muscle injury unrelated to this study, and so did not cycle with test conditions involving overlap reduction. P01 and P02 cycled with the listed stimulation parameters during all test conditions involving low overlap. Note that stimulation through one contact in each leg of P01 resulted in high overlap with other contacts with little additional muscle output at output levels required for cycling, so it was excluded from the low overlap stimulation pattern.

TABLE 2
Stimulation parameters used during low overlap test conditions and
the resulting overlap reductions, presented as the mean ± standard
deviation of the difference in overlap between optimized and maximum
stimulation levels for each pairwise combination of contacts

| | | Low Overlap Stimulus Level [us] | | | Overlap |
|---|---|---|---|---|---|
| Participant Leg | | Contact 1 | Contact 2 | Contact 3 | Reduction |
| P01 | Right | 175 | 105 | 0 | 21.2 ± 3.2% |
| | Left | 105 | 0 | 95 | 30.4 ± 1.1% |
| P02 | Right | 165 | 160 | 150 | 16.8 ± 5.9% |
| | Left | 145 | 105 | 100 | 18.8 ± 8.0% |

Carousel with Maximum Overlap (C-Max) rotates supramaximal stimuli (PW=255 μs) through independent contacts to maximally activate a different MUP each pedal stroke, reducing duty cycle. P01 performed carousel cycling trials involving all three contacts (C-Max 3c) as well as just the two strongest contacts per leg (C-Max 2c), as one activated fiber pool per leg was significantly weaker than the others. This enabled insight into the trade-off between keeping duty cycle as low as possible by including a weak group and having less duty cycle reduction but only relying on the strongest fibers. Following observations from P01, carousel stimulation trials with the remaining participants were set to involve only their strongest MUPs. Therefore, for P02, three MUPs on the right leg and only the two strongest MUPs on the left leg were used. For P03, only the two strongest MUPs on each leg were used. Carousel, Low Overlap (C-Low) combines low overlap and low duty cycle approaches by activating one MUP per pedal rotation with optimized stimulus values.

Each experimental session began with a warm-up trial with stimulation followed by a S-Max stimulation trial to obtain baseline performance metrics with their usual cycling pattern. Subsequent trials alternated stimulation conditions between one of the test conditions and S-Max. Exercise trial durations were determined based on the amount of time the participant could continuously cycle with the conventional S-Max stimulation pattern (5, 2, and 1.5 min for P01, P02, and P03 respectively). Around their respective maximal trial times, P02 and P03 consistently paused at various points of the pedal stroke and needed to push their leg with their arm to continue, prompting cutoff at those durations. P01 could cycle well beyond 5 min, but time limitations prompted us to end trials when power output typically reached a steady state. Though trial durations varied by participants based on ability level, trial durations were kept consistent across simulation conditions within subjects. To prevent any cumulative effects of fatigue from influencing the results, rest breaks at least double each participant's respective active cycling times (i.e., at least 10, 4, and 3 min for P01, P02, and P03 respectively) were imposed between trials.

### Outcome Measures and Statistical Analysis

Cycling performance metrics were measured by a Garmin Edge bike computer (Garmin Ltd., Olathe, KS) communicating with Quarq DZero power crank arms (SRAM LLC, Chicago, IL). Total work (W) was calculated as cycling power output integrated over trial duration. Increased W indicates greater exercise intensity was maintained throughout the trial, as trial durations were equal across stimulation conditions within each participant. End power (Pend) averaged the power output over the final third of each trial to approximate steady state power output. Higher Pend indicates that a stimulation condition improves steady state power maintenance. W and Pend are presented as the differences in work and end power (ΔW and ΔP_{end} respectively) between test condition and S-Max trials from the same experimental session. A power fluctuation index (PFI) was calculated over each 6 s window to encompass several full pedal revolutions to characterize the smoothness of power production between pedal strokes for each stimulation condition. To calculate PFI, a linear least squares line was fit to the raw power data within each window to establish a local trend. The maximum and minimum difference between raw power and the fitted line were used to calculate a power deviation range around the general trend in each window. This range was then divided by the average of the trend for the PFI of that window. Examining the fitted trend within each window ensures steady decreases in power due to fatigue are not interpreted as large differences between pedal strokes. Lower PFIs indicate more consistent power outputs and smoother rides. Charge accumulation (Q) was calculated as the integral of the pulse amplitude multiplied by pulse width over time to characterize differences in stimulation efficiency (Δη) between S-Max and test conditions:

$$η = \frac{W}{Q} - \frac{{W}_{S - Max}}{{Q}_{S - Max}}$$

Each participant performed a minimum of six trials per test condition, each with a corresponding number of conventional stimulation trials for comparison. Homogenous (Levene's test for equal variances p>0.05), though sometimes non-normal (Shapiro-Wilks test for normality p<0.05) W and Pend data sets prompted application of Mann-Whitney tests for nonparametric comparison of two independent samples (test condition trial outcomes vs. corresponding conventional stimulation trial outcomes). PFI data sets were found to be neither homogenous nor normally distributed. PFI were thus compared among all stimulation conditions using Welch's ANOVAs followed by post-hoc Dunn's tests using Matlab's multcompare function. Welch's ANOVAs do not assume equal variances and are robust to deviations in normality with sufficient sample size (n>180 for all conditions and participants).

### Results

### Total Work and End Power

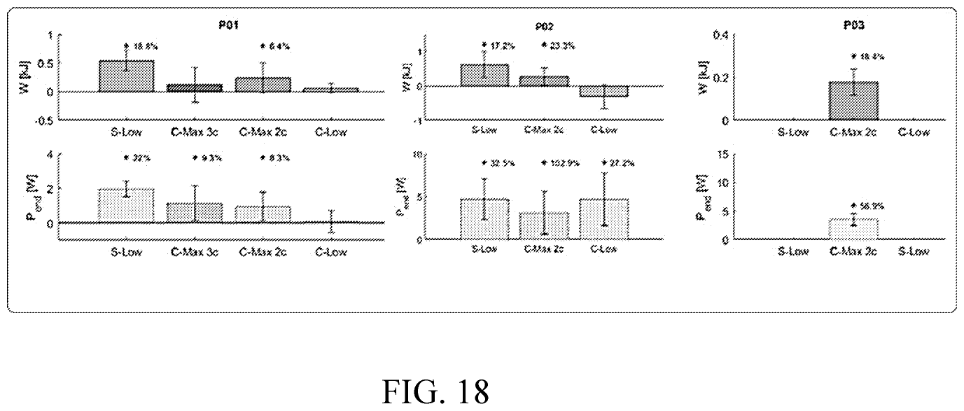

W and Pend were examined as the differences in outcomes from trials of a given test condition and S-Max trials from the same experimental session and are shown in FIG. 18. FIG. 18 shows differences in W and Pend for each test condition compared with S-Max stimulation for P01 (left), P02 (middle) and P03 (right). Percent increase is listed for differences with statistical significance (p<0.05). Positive ΔW values indicate that the test condition enabled more work to be performed than conventional stimulation in the same amount of time. Positive ΔP_{end} values indicate a test condition increased steady state power maintenance over conventional stimulation. Significant positive ΔW and ΔP_{end} were found for various test conditions in all participants. For conditions with statistical significance, percent improvement relative to S-Max outcomes are also included in FIG. 18. P01 performed significantly more work after 5 min of cycling with S-Low (18.8%, p<0.05) and C-Max 2c (6.4%, p<0.05) stimulation than with conventional (S-Max) stimulation. P01 also showed a trend of slightly increased W with C-Max 3c and C-Low, though these differences were not significant. Except for the C-Low condition, all test conditions maintained significantly higher end powers than conventional stimulation (22.0%, 9.3%, and 8.3% for S-Low, C-Max 3c, and C-Max 2c respectively, p<0.05).

P02 performed significantly more work than S-Max after 2.5 min of cycling with S-Low stimulation (17.2%, p<0.05) and C-Max 2c (23.3%, p<0.05). W decreased with C-Low stimulation, though not significantly. P_{end} was significantly higher than S-Max for all test conditions (S-Low 32.5%, C-Max 2c 102.9%, and C-Low 27.2%, p<0.05). Note that percent improvements were greater for C-Max 2c despite the absolute value of the differences being lower than with the other stimulation methods. This was due to a large decrease in baseline cycling ability from an extended period of inactivity during the COVID-19 pandemic before C-Max 2c could be tested. Thus, while the absolute value of W and Pend improvements were smaller when using the C-Max 2c pattern, they actually reflected larger improvements relative to conventional stimulation trials performed at that same point in time.

P03 performed significantly more work (18.4%, p<0.05) with C-Max 2c stimulation after 1.5 min of cycling compared with S-Max stimulation. End Power was also significantly greater (56.9%, p<0.05) with C-Max 2c stimulation.

### Power Fluctuation

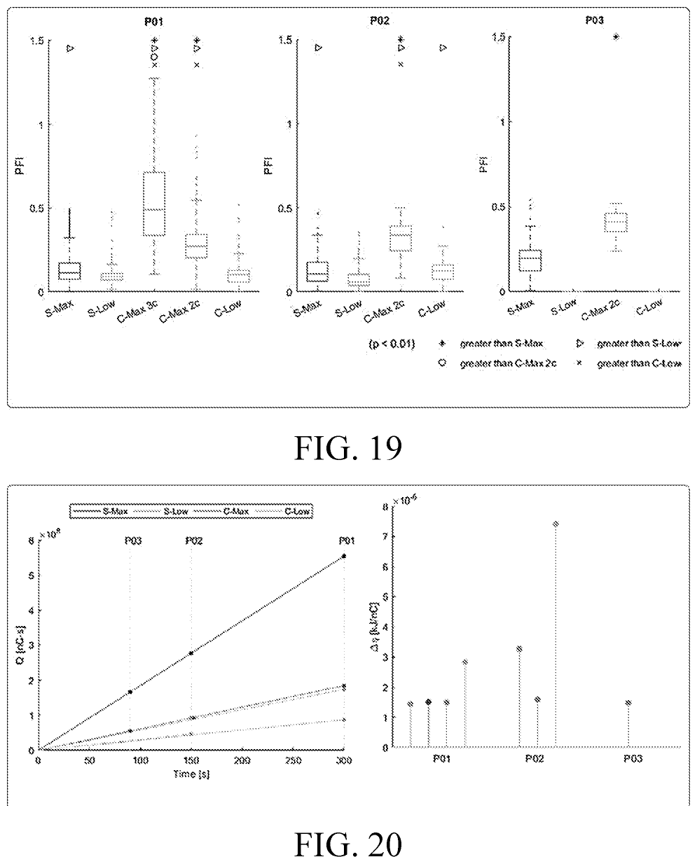

PFI varied among stimulation conditions (FIG. 19). FIG. 19 illustrates Power fluctuation indices of each stimulation condition for P01, P02, and P03. Lower PFI values indicate lower stroke-to-stroke variability in power output while pedaling and a smoother ride. In all participants, conventional S-Max stimulation resulted in a median PFI below 0.2, meaning less than 20% fluctuation in power typically occurred over several revolutions. S-Low caused a significant PFI decrease relative to S-Max in P01; in P02 no significant difference was found. C-Max patterns with all three contacts increased PFI significantly (p<0.01) compared with S-Max stimulation in P01, resulting in a PFI median of 0.49 and maximum of 2.7. C-Max with only the two strongest contacts significantly reduced PFI to a median of 0.27 and a maximum of 1.4 in P01, but this was still significantly greater than the S-Max condition. C-Max involving only the strongest contacts similarly resulted in PFIs significantly higher than S-Max with medians of 0.33 and 0.41 for P02 and P03 respectively. C-Low increased PFI slightly relative to S-Max in P02, while in P01 no significant difference was found.

### Charge Accumulation and Efficiency

All selective stimulation conditions injected less charge throughout the cycling trials than the S-Max pattern (FIG. 20). FIG. 20 shows: (LEFT) charge accumulation over time for each stimulation condition. Dots represent total charge injection at the end of each participant's trial length, indicated by the vertical dotted lines. Low overlap and/or low duty cycle test conditions inject much lower Q than conventional stimulation; (RIGHT) difference in stimulation efficiency compared with S-Max for each test condition and participant. Positive efficiency differences indicate selective patterns resulted in more work per unit of charge injected. C-Low has the lowest Q accumulation as it combined both low overlap and low duty cycle stimulation approaches. S-Low and C-Max had similar Q accumulations that, while higher than C-Low, are still considerably lower than S-Max. All test conditions in all participants resulted in positive Δη, producing more work per unit of charge than with conventional stimulation. C-Low, despite producing insignificant changes in work compared with S-Max, resulted in the highest efficiencies due to the pattern's extremely low Q (FIG. 20).

### Discussion

### General Outcomes

For all participants, at least one stimulation condition significantly improved functional outcome measures over conventional stimulation, demonstrating that selective neural stimulation methods can improve exercise performance after SCI. Though differences exist among participants and patterns, a general discussion for trends in each outcome measure and their implications is first presented in the following sections

### Total Work

S-Low and C-Max 2c paradigms significantly increased total work performed over conventional (S-Max) stimulation, effectively enabling a more intense workout within the same amount of time. Studies have shown that cycling intensity, rather than duration, has a significantly greater influence on predictors of future health, and that higher intensity cycling workloads result in greater improvements in leg strength, body composition, cholesterol levels, and blood pressure compared with longer exercises at low intensities. Furthermore, enabling participants with SCI to achieve more work in less time may improve exercise regimen satisfaction and adherence.

Unlike the other test conditions, C-Low did not result in significantly different work compared with conventional stimulation. In participant P02, C-Low seemed to decrease work performed in the same amount of time, though not significantly. These results are unsurprising as C-Low delivers the lowest amount of stimulation to the fewest MUPs out of all the patterns tested. The peak power produced at the beginning of C-Low trials was often much lower than with other stimulation patterns, causing work, the integral of power over time, to accumulate much more slowly. It is contemplated that C-Low can be retained as a viable stimulation option, though, because it provides other benefits discussed below.

### End Power

S-Low and C-Max patterns also significantly increased end power, suggesting they could continue to outperform conventional stimulation in longer cycling trials. Even C-Low, which did not produce significant increases in work, still resulted in significantly higher end power in one participant. In this case, significant work increases may have been seen if trial durations were sufficiently long. Trial durations were limited in this study due to time constraints and the desire to prevent irrecoverable fatigue within a single session, but could be readily extended in future work. Higher end powers also indicate that more force was produced and maintained by the muscles. Greater force production can enable cycling against higher resistances, which may further accelerate strength gains and enable more consistent improvements in load-dependent biomarkers, such as bone density, with these systems.

### Power Fluctuation

The power fluctuation index represents the smoothness of a cycling stimulation pattern and is an important consideration for balanced muscle loading and user comfort. No significant difference in PFI was found between S-Low and S-Max in P02, and S-Low significantly decreased PFI relative to S-Max in P01. These results show that cycling smoothness is not compromised by overlap reduction, and in some cases may even be improved. This improvement may be attributed to more balanced force output among the left and right legs at optimized stimulation levels.

C-Max patterns did result in significantly increased PFI and thus greater instability in all participants. This was anticipated since carousel cycling, or any pattern that involves unsynchronized activation of multiple MUPs, risks uneven force production among the different fiber groups. At maximum stimulation levels, the fiber groups activated by the different electrode contacts produced a wide range of power outputs that resulted in some quick and strong revolutions interspersed with slower, weaker ones. The difference in power output among revolutions is reflected in the higher PFI values for C-Max conditions and manifests as a somewhat erratic, jerky cycling motion. This choppiness was most prevalent at the beginning of the carousel trials but subsided as each MUP eventually fatigued, presumably to more consistent levels. For P01, the C-Max pattern involving all three contacts (C-Max 3c) caused occasional pauses in cycling as one fiber group per leg was substantially weaker than the others even when maximally activated. For that reason, C-Max with only the two strongest contacts per leg (C-Max 2c) was also studied. This modified C-Max pattern significantly reduced PFI compared with the three contact version. The increased stability and elimination of pauses in pedaling likely contributed to the significant increase in work with C-Max 2c but not C-Max 3c, as carousel stimulation is always limited by the weakest muscle group activated. There appears to be a clear trade-off between reducing duty cycle to prolong output as much as possible and reducing the overall strength and stability of the pattern as a whole. From these results, it appears that a slightly higher duty cycle with significantly less power fluctuation outperforms the lowest possible duty cycle that exhibits substantial instability. PFI is thus an important area of improvement for carousel cycling so that users may take full advantage of the benefits of low duty cycle without large power variations. Using only the strongest MUPs within the carousel pattern still produced significantly higher PFIs compared to conventional stimulation in all participants, but these power fluctuations were not great enough to make the pattern unusable or be perceived as uncomfortable or distracting.

The C-Low pattern reduced PFI significantly relative to C-Max, even to the point of not being significantly different than S-Max in one participant. This improvement is again likely attributed to more balanced output among fiber groups at optimized stimulation values. However, the powers produced by the few fibers recruited each revolution, while balanced, could not reach nearly the same peak powers as conventional stimulation and often resulted in consistently smooth but weak cycling. It is contemplated that future studies can look to control each contact's stimulation level throughout a trial to maintain a stable yet strong target power. This would ensure the high stability benefits of C-Low cycling while also gradually recruiting more fibers as necessary to achieve the improved work benefits of C-Max.

### Charge Injection and Efficiency

Selective stimulation patterns, by design, inject less charge over time because of their optimized stimulus levels and/or reduced duty cycle. S-Low and C-Max patterns produced more work with less charge than conventional stimulation, and therefore also had higher efficiencies. Even C-Low, which did not differ significantly in work from S-Max, still resulted in more efficient stimulation as it utilized both low overlap and low duty cycle strategies for very low charge injection. Higher efficiency can prolong battery life of the stimulation control units, which is extremely important for practical, everyday use of these systems. Furthermore, reducing the amount of charge needed to produce a desired exercise or motion can greatly decrease the risk of overstimulating and thus minimize any potential of damaging the neural tissue over time.

### Pattern-Specific Discussion

Pattern-specific considerations and differences in performance among participants are addressed in the following sections.

### Low Overlap Stimulation

S-Low stimulation-induced cycling was successful for both participants who underwent the stimulation level optimization procedures. Overlap reductions of at least 16% significantly increased total work and end power. In fact, these overlap reductions resulted in some of the largest percentage increases in work performed over conventional stimulation out of all patterns tested in both participants. Further reductions in overlap could extend these advantages, though care must be taken not to drastically reduce stimulation levels to minimize overlap at the expense of adequate muscle output.

Even prior to tuning the system pulse parameters to minimize motor unit overlap in this study, the three out of eight possible femoral nerve C-FINE contacts had been chosen specifically for P02's conventional stimulation patterns due to their high selectivity for independent knee extensor muscles. Improvements in P02's performance with S-Low stimulation thus indicates that even already highly selective systems can still be further improved with overlap optimization. P01 has a slightly different stimulation system which is thought to be inherently less selective as there are fewer, larger contacts within the spiral nerve cuff electrodes that may more readily activate the same neural fibers. This is reflected in the fact that one contact per leg was left out of the pattern during optimization, and that the pulse width values needed for low overlap in P01 were considerably lower than those used for P02. Because of the greater initial overlap with conventional stimulation, a greater percent reduction was able to be achieved for P01 than P02, which corresponded with an even greater percent improvement in work. Though this case series does not attempt to make direct comparisons between subjects due to differing ability levels and stimulation systems, the data do support the idea that greater reductions in overlap could result in greater improvements in functional outcome. It also highlights the particular importance of reducing overlap in stimulation systems that do not have a high initial degree of selectivity.

### Low Duty Cycle Stimulation

C-Max cycling results agree with other studies of similar duty cycle reduction techniques to improve functional outcomes during isometric contractions. Improvements with duty cycle reduction are often partially credited to the pumping action that is created when activation is rotated among different fiber groups, which can promote blood flow and oxygen delivery to the muscle. However, as this exercise is cyclic in nature and already comprised of on-off activation patterns within each leg that promote blood flow, it is likely not the main contributor to the success of the carousel stimulation pattern. The carousel stimulation scheme activates each fiber group less often, which can delay glycogen store depletion. The longer rest periods each fiber group experiences can also encourage more complete clearance of metabolite build-up prior to the next contraction. Together, those two benefits may be more likely to account for improved work and power maintenance with C-Max stimulation-induced cycling.

To the authors' knowledge, only one other study has used a carousel stimulation scheme during a functional cycling task for people with paralysis. That study alternated activation between the rectus femoris and medial/lateral vasti muscles several times within each pedal stroke, as opposed to the one fiber pool per pedal stroke in this study. It demonstrated that an alternating stimulation strategy enabled rides of longer duration and distance while maintaining the same mechanomyogram amplitude in each muscle group. However, no direct power or work measurements were reported and no power fluctuation analyses were included. Furthermore, that study employed two different sized surface electrodes with slightly different placements between the standard and alternating conditions, and did not conduct preparatory overlap analyses to ensure separate fiber populations were being activated in each case. This creates uncertainty as to whether the reported endurance gains were from activation of a different muscle mass or truly from the reduction of duty cycle. In the study, implanted electrodes ensured that the relationship between the contacts and the nerve fibers remained consistent between conditions such that the only changing variable was the stimulation pattern. A priori, overlap analyses also ensured the selectivity of independent motor unit pools, and power fluctuation analysis provided insight into the effects that stimulus modulation strategies have on ride smoothness and user comfort. The study was clearly able to demonstrate that activating the exact same groups of independent fibers at a lower duty cycle is sufficient to significantly increase sustained exercise intensity.

One suggested advantage of more frequent alternations in the study by Decker et al. is that only the first fiber group activated for each pedal stoke must overcome the passive resistance of the muscle and tendon, thus requiring less effort from the subsequently activated fibers within that revolution. Future studies with implanted electrodes may look to similarly alternate stimulation among multiple contacts within each pedal stroke to take advantage of this. By overcoming the initial resistance to movement with the strongest fiber pool, weaker fiber pools may be able to finish the pedal stroke with minimal effort. This could enable fiber pools that are too weak to effectively cycle on their own, like one observed in each leg of P01 and P03, to effectively contribute to cycling and further improve W and Pend measures. However, switching stimulation through various contacts within a pedal stroke may cause unwanted increases in instability and fiber overlap. This is because the sequential ramping up and down of stimulation between contacts during one pedal stroke can have to overlay in such a way to prevent dead spots and pauses. This could cause some fiber groups to be activated before another has fully relaxed, leading to sudden leg jerks and potentially contributing to fiber overlap while two contacts are briefly delivering stimulation at the same time.

### Combined Low Overlap, Low Duty Cycle Stimulation

C-Low stimulation results varied the most out of all patterns tested. Average work increased in P01 and decreased in P02, though neither were significant. End Power was significantly increased in P02 and largely unchanged in P01. It is possible that C-Low's success during cycling depends on a participant's strength and absolute power output capabilities. P02 had power output peaks over four times that of P01 during conventional cycling (80 watts vs 18 watts) but had much lower endurance (2.5 vs 5 min continuous cycling times) due to rapid power decline. C-Low stimulation enabled P02 to cycle approximately 5 watts higher at steady state but achieved a considerably lower peak power compared with conventional stimulation, resulting in significantly higher end power but not total work. With extended trial durations, participants with higher power outputs like P02 and P03 could benefit from C-Low as substantial power is still possible even with the small fiber activation typical of a fatigued state. Participants with much lower power out-puts, like P01, may be unable to extend trial durations to the point of significant work improvements as the few fibers activated likely may not produce enough power over time to continue cycling. C-Low may thus be better suited as a short strength building exercise pattern for these weaker participants to specifically target and strengthen individual fiber pools.

### Example 5

People with spinal cord injury (SCI) or other neuromuscular disorders are at high risk for secondary health issues due to immobility from lost volitional muscle control. Electrically-induced cycling can engage paralyzed musculature in exercise to prevent or mitigate some of these health issues. This technology has been shown to improve muscle mass, circulation, body composition, and quality of life with continued use. However, such improvements often develop slowly as rapid muscle fatigue is common with these systems and greatly reduces sustained exercise intensity and endurance within a single session. Additionally, improvements in physiological factors that are load dependent, such as bone density, are not yet well established because the limited sustained force production prevents prolonged cycling against sufficient resistances.

Recent findings have shown significant improvements in exercise ability with selective stimulation strategies. Reducing either the duty cycle or the relative frequency of stimulation of the activated knee extensor musculature significantly increased work performed and power maintained over conventional, supramaximal stimulation within a cycling exercise session. Firing frequency can be reduced by applying submaximal levels of stimulus through independent electrode contacts to activate non-overlapping motor unit pools (MUPs). Lowering stimulus levels to prevent the electric fields of nearby electrode contacts from overlapping avoids over-stimulating the common fibers that can otherwise be within the overlapping regions. Though lower stimulus levels led to lower initial peak powers, preventing over-stimulation enabled higher steady state powers to be maintained for longer periods of time, resulting in increased accumulated work. Reduced duty cycle, or “carousel” stimulation, on the other hand rotated activation among the independent knee extensor fiber groups by stimulating through a different contact each pedal rotation. This once again lowered peak power output as fewer fibers were active at a time, but the longer recovery periods between successive contractions of the same group improved power maintenance and increased total work performed. These strategies were largely successful in an open-loop implementation, although carousel stimulation sometimes exhibited significant power fluctuations due to variations in the stimulated strength of each independent MUP. These fluctuations due to uneven force production among independent fiber groups caused pedal strokes to vary in strength and speed.

Another strategy that may address the variability in contraction strength and further improve exercise performance after paralysis is closed-loop feedback control of the stimulation intensity to maintain a consistent, but submaximal level of power output. Open-loop stimulation-induced cycling programs employ preset, and often supramaximal, levels of stimulation throughout the exercise. Unlike volitional exercise in able-bodied individuals that stochastically activates only the motor units required to maintain a desired intensity, such approaches continuously activate many motor units, precluding energy savings that could otherwise be harnessed later in the cycling session. Instead, modulating stimulation levels from initially low overlap values with feedback control has the potential to maintain submaximal exercise intensity at a higher steady state output. Although this can again result in a lower initial peak intensity, adjusting stimulation as needed to recruit not-yet-fatigued fibers can maintain a mid-level intensity for longer and ultimately improve endurance and produce more work within an exercise session. This can also address the power fluctuation issues when combined with duty cycle reducing stimulation patterns by ensuring each fiber group produced similar outputs to match a steady target value when active.

Several research groups have previously investigated closed-loop cycling strategies for cadence control. These studies largely focus on controlling the output of an external motor incorporated into the cycling apparatus to compensate for weak muscle output and rapid muscle fatigue. Motor output is used to resist when muscle contractions are strong and assist when they become too weak. This ensures constant cadence despite varying muscle states, but does not maintain submaximal contractile effort or conserve energy to prolong cycling duration. Providing motor assistance to achieve target cadence can decrease effort required of the muscles prematurely and shield the paralyzed legs from positive stresses that could further benefit muscle and bone health. The present study therefore sought to maximize impact on the paralyzed musculature by implementing closed-loop control of electrical neural stimulation only on a motorless recumbent trike. It was investigated that the relative performances of open-loop cycling with a fixed knee extensor stimulation intensity, closed-loop modulation of synchronous activation of all available knee extensor MUPs, and closed-loop carousel stimulation that modulated activation of a different independent MUPS during each successive pedal stroke in participants with chronic paralysis due to SCI or other upper motor neuron dysfunction. It is contemplated that closed-loop control can improve endurance in terms of end power output and work performed, reduce power fluctuations, and increase efficiency in terms of output per unit charge within an exercise session over conventional open-loop stimulation patterns. It is further contemplated that functional improvements can correlate with positive impacts on physiological responses to exercise.

### Methods

### Experimental Setup and Protocol

Six participants with paralysis performed controlled cycling trials. Each had previously received an implanted neural stimulation system to activate the otherwise paralyzed musculature of the trunk and lower extremities. The implanted systems and electrode variations used in biking are described elsewhere. A summary of participants' paralysis classifications and relevant implanted electrodes is provided in Table 3.

TABLE 3
Summary of participants and implanted stimulation
system knee extensor contact details.

| | Paralysis Cause/ | Knee Extensor Stimulation |
|---|---|---|
| Participant | Classification | (Independent Contacts) |
| P01 | C7 AIS-B SCI | Proximal femoral spiral cuffs (2) |
| | | VL epimysial (1) |
| P02 | T10 AIS-A SCI | Proximal femoral C-FINEs (3) |
| P03 | C7 AIS-C SCI | Proximal femoral C-FINEs (3) |
| P04 | C7 AIS-C SCI | Proximal femoral spiral cuffs, |
| | | single channel (1) |
| P05 | T3 AIS-A SCI | Proximal femoral spiral cuffs, |
| | | single channel (1) |
| P06 | Adult Onset | Proximal femoral spiral cuffs, |
| | Leukodystrophy | single channel (1) |

Participants were seated on a recumbent bike (Catrike, Orlando, FL) with their legs secured in custom orthotics affixed to the pedals (FIGS. 4 and 7A). A crank angle encoder (US Digital, Inc.) relayed instantaneous pedal position to an external control unit (ECU) running a custom stimulation model designed in Simulink. Within the stimulation model, pedal position is mapped to the appropriate muscle activations and corresponding stimulus patterns that produce smooth cycling based on able bodied and surface stimulation cycling literature. Muscle activation timing patterns were further customized heuristically with participants in the loop to adjust for each individual's physical characteristics and implanted system capabilities. The ECU relays the desired stimulus parameters (pulse amplitude, pulse duration and stimulus channel) based on crank angle to the implanted pulse generator via external radiofrequency coil. The pulse generator then delivers stimulating current through various implanted electrode contacts on or near the peripheral nerves to activate the paralyzed musculature and induce the cycling movement.

Electrode contacts that activate the quadriceps, hamstrings, hip adductors, and gluteal muscles may all be involved in cycling exercise patterns. For this study, only stimulation to the quadriceps (knee extensors) activating contacts differs among the three stimulation conditions tested. The conventional open-loop stimulation pattern, Standard Max (S-Max), delivered supramaximal stimuli through all knee extensor-activating electrode contacts each pedal rotation. Standard controlled (S-Cont) stimulation also delivered stimulation through all knee extensor contacts each pedal rotation while modulating pulse duration to all simultaneously on each side, while carousel controlled (C-Cont) stimulation rotated activation among independent knee extensor fiber groups by modulating stimulation through a different contact each pedal stroke. Both controlled patterns began at submaximal stimulus levels and used PI controllers within the model to adjust PW values and maintain a desired cycling cadence (FIG. 2). Differences between actual cycling cadence and target cadence created an error signal e (t) that drove the PI controller(s) to adjust pulse width (PW) sent through the active contact(s). PI gains were initially set according to those found to best track a sinusoidal target output during isometric studies and further heuristically adjusted as necessary to produce smooth but responsive cycling.

Instantaneous cadence was calculated in the stimulation model as the rate of change of the crank angle. Because cadence is used as a feedback signal, which is proportional to a different power output for each gear on the drivetrain, the participant remained in the same gear throughout these cadence-controlled trials to ensure actual power was in the desired mid-level intensity range. Controlled trial fixed gears were chosen to be between the largest and smallest gear participants shifted through during S-Max trials and target cadence was chosen such that estimated power output can be between peak and steady state from those trials on the chosen gear.

Each experimental session began with a short warm-up trial with stimulation followed by an S-Max trial to assess baseline performance. Subsequent trials alternated between a controlled condition and S-Max with breaks at least double the cycling duration between trials to prevent cumulative fatigue. Exercise trial durations were 5, 2.5, 1.5, 3, 2.5, and 2 minutes for participants 1-6 respectively, and were determined based on the amount of time the participant could continuously cycle with S-Max. Around their respective trial times, P02, P03, and P06 can consistently pause at various points of the stroke cycle and may need to assist knee extension by pushing on their thighs with their arms to continue, prompting cutoff at those durations. P01, P04, and P05 could cycle well beyond their trial lengths, but time limitations prompted us to end trials when S-Max power output typically reached a steady state. Though trial durations varied by participant based on ability level, they were kept consistent across simulation conditions for each subject.

### Outcome Measures

A Garmin Edge bike computer (Garmin Ltd., Olathe, KS) communicating with Quarq DZero power crank arms (SRAM LLC, Chicago, IL) provided functional cycling outcome measures. Total work (W) was calculated as cycling power output integrated over trial duration. Increased W indicates greater exercise intensity was maintained throughout the trial. End power (Pend) averaged the power output over the final third of each trial. Higher Pend indicates that a stimulation condition improves steady state power maintenance. A power fluctuation index (PFI) was calculated as the mean ratio of peak-to-peak power relative to the de-trended average power over each 6 second window to encompass several full pedal revolutions. A lower PFI indicates a more consistent power output and smoother ride. Root-mean-squared error (RMSE) was calculated for controlled conditions to determine how well a target cadence was maintained by a given controller configuration.

Initially, trials were performed in the Motion Study Laboratory at the Louis Stokes Cleveland VA Medical Center. In-laboratory trials ran the custom Simulink cycling models through MATLAB real-time xPC target, from which controller PW outputs could be accessed and analyzed. Total charge injection (Q) was calculated as the sum of the integrals of PW through each contact over time, and was used to calculate a stimulation condition's efficiency (η) as W/Q. Higher η indicates a condition produces more work per unit of charge injected. Due to the COVID-19 pandemic and subsequent pause on in-person research, several cycling sessions were performed remotely by participants who already had recumbent cycling setups at their own homes. In these cases, stimulation models were compiled into standalone ECUs. This enabled successful deployment and testing of various controller patterns remotely, but did not allow collection of controller output due to ECU storage limitations, thus preventing analysis of Q and n in these cases.

Two physiological metrics were also assessed with select participants. MOXY muscle oxygenation monitors (Fortiori Design, LLC, Hutchinson, MN) measured the muscle oxygen saturation (SmO2) of various activated heads of the quadriceps in three participants through near-infrared spectroscopy. SmO2 is the ratio of oxygenated hemoglobin and myoglobin to total hemoglobin and myoglobin in the underlying muscle tissue, and provides insight into the relative delivery and extraction of oxygen within a specific region of muscle fibers. Declining SmO2 values indicate the muscle fibers are utilizing oxygen faster than they are being supplied, and that an exercise intensity is likely not sustainable under current conditions. Lastly, heart rate was monitored during select trials of S-Max and S-Cont cycling with one participant using a Garmin Vivosmart (Garmin Ltd., Olathe, KS) wrist-worn activity tracker. This was done to determine if any resulting functional improvements in cadence-controlled cycling performance can be sufficient to evoke corresponding changes in heart rate, which is relatively unresponsive to stimulation-induced lower extremity cycling in participants with paralysis, particularly those with lesions above the Tl level.

### Statistical Analyses

Unless otherwise noted, participants completed at least six trials of a cadence-controlled stimulation condition and a corresponding number of conventional stimulation trials for comparison. Mann-Whitney nonparametric tests were applied to W and Pend data since the results were independent, homogenous (Levene's test for equal variances p>0.05), but non-normal (Shapiro-Wilks tests for normality p<0.05) samples. PFI and heart rate data sets were compared using Welch's ANOVAs followed by post-hoc Dunn's tests.

### Results

### Functional Outcomes: Total Work, End Power, and Power Fluctuation

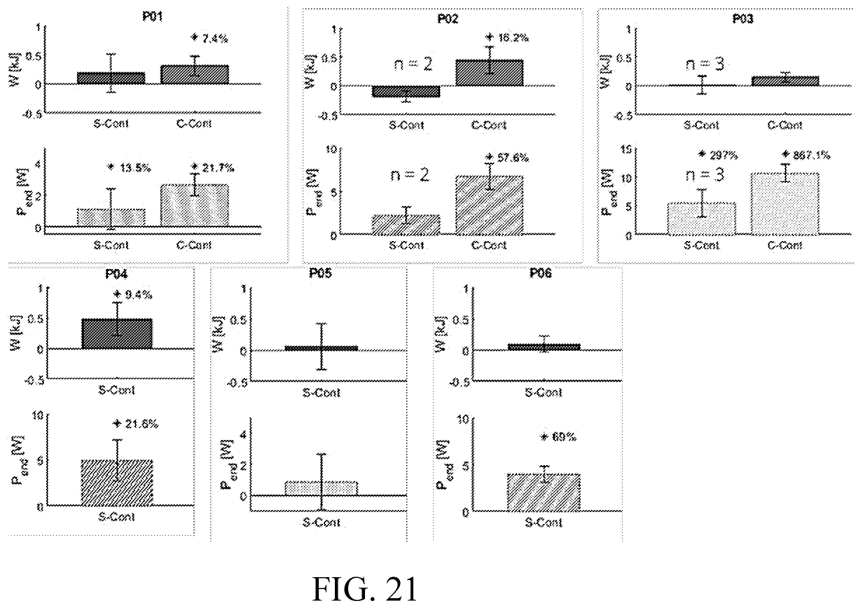

Differences in W and Pend outcome measures between trials of a given controlled test condition and S-Max stimulation trials are presented in FIG. 21. Positive differences indicate that a controlled condition improved total work performed or power maintained over conventional stimulation within the same trial duration. Increased ΔW suggests a more intense bout of exercise. Increased ΔP_{end} demonstrates greater achieved endurance as higher power was maintained through the end of the trial. While absolute differences in W and Pend varied substantially among participants and between conditions, controlled stimulation conditions consistently tended to improve work and end power outcome measures, though not always significantly, over conventional stimulation.

S-Cont stimulation significantly increased Pend in four out of the six participants tested (P01: 13.5%, P03: 297%, P04: 21.6%, and P06: 69%), but produced a significant improvement in W in only one participant (P04: 9.4%). All other participants saw no significant difference in work between S-Cont and S-Max. C-Cont stimulation significantly increased Pend in all three participants tested with the low duty cycle controlled condition (P01: 21.7%, P02: 57.6%, P03: 867.1%). C-Cont stimulation also significantly increased W for two of those participants (P01: 7.4% and P02: 16.2%). The third participant saw no significant different in work between C-Cont and S-Max.

FIG. 21 shows difference in W and Pend between controlled stimulation conditions and S-Max stimulation trials. Positive differences indicate the test condition improved work and end power maintenance compared with conventional, open-loop cycling. Percent improvement is given for differences with statistical significance (p<0.05). Note that participants completed at least six trials of cadence-controlled conditions and a corresponding number of S-Max trials, except where lower n values are indicated.

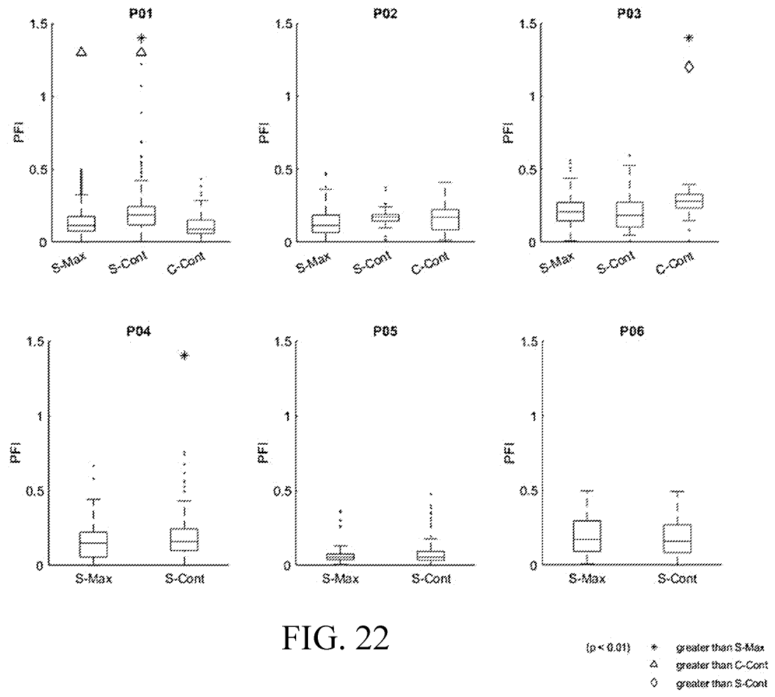

Measurements of PFI resulting from S-Max and controlled stimulation conditions are presented in FIG. 22. Feedback control significantly reduced PFI relative to open-loop low duty cycle approaches, but remains significantly higher than S-Max stimulation in three participants.

FIG. 22 shows Power fluctuation indices (PFI) for conventional and cadence controlled stimulation conditions. Lower PFI indicates a smoother, more stable ride.

### Controller Performance: RMSE, Charge Accumulation, and Efficiency

Absolute RMSE and RMSE as a percentage of target cadence were calculated for each participant and controlled stimulation condition (Table 4). Target cadences ranged from 25-52 rpm. Average RMSE and RMSE % ranged from 1.1-3.7 rpm and 3.4-10.5% respectively, indicating good controller tracking performance prior to reaching maximum allowed stimulus levels due to advanced fatigue.

TABLE 4
Controller target tracking performance
for controlled stimulation conditions.
RMSE is calculated only for the portion of the
trial where the controller is actively adjusting PW,
before reaching maximum due to progressive fatigue.
Ranges of target cadences tested with participants
are presented where applicable. Lower RMSE and RMSE %
indicates better tracking performance.

| | | Target Cadence | RMSE | |
|---|---|---|---|---|
| Participant | Controller Type | Range [rpm] | [rpm] | RMSE % |
| P01 | Standard | 40-44 | 2.3 ± 1.6 | 5.6 ± 3.6 |
| | Carousel | 33 | 1.1 ± 0.4 | 3.4 ± 1.3 |
| P02 | Standard | 32 | 2.8 ± 0.7 | 8.6 ± 2.3 |
| | Carousel | 42 | 1.6 ± 0.4 | 3.9 ± 0.9 |
| P03 | Standard | 45-50 | 2.5 ± 1.4 | 5.1 ± 2.7 |
| | Carousel | 35-38 | 3.7 ± 1.5 | 10.3 ± 4.6 |
| P04 | Standard | 45-52 | 1.8 ± 0.9 | 3.8 ± 1.8 |
| P05 | Standard | 25-50 | 2.3 ± 0.9 | 6.6 ± 2.1 |
| P06 | Standard | 28-44 | 3.5 ± 2.0 | 10.5 ± 6.1 |

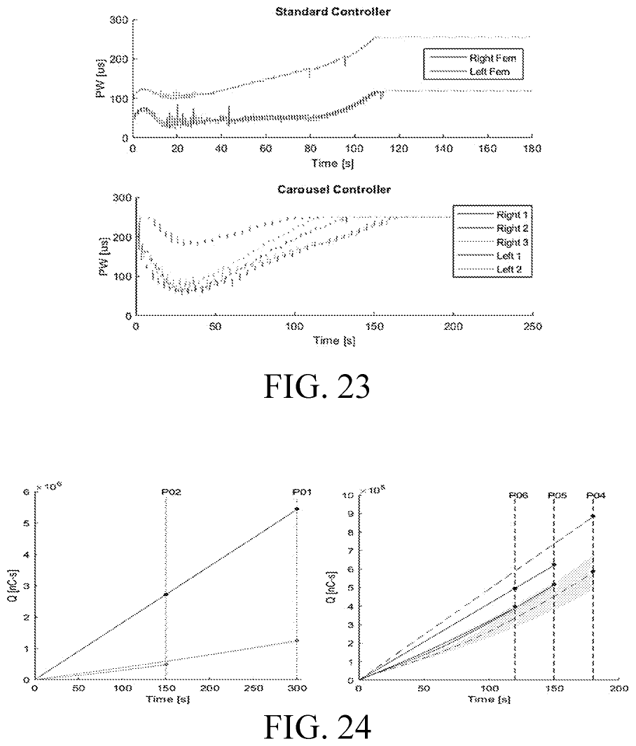

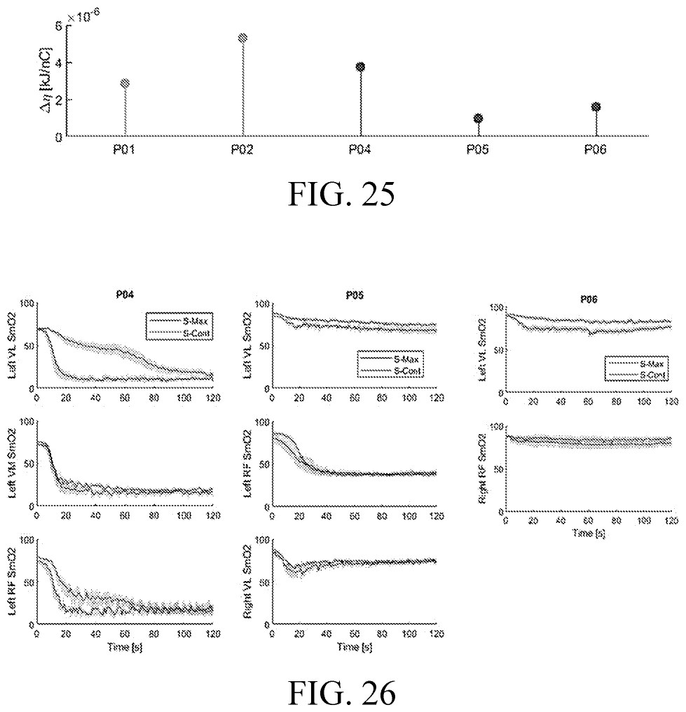

Controller PW output values saved from in-laboratory trial sessions enabled post-hoc analysis of charge accumulation and efficiency. Stimulus levels were dynamically adjusted by the controllers to account for both muscle potentiation and fatigue (FIG. 23). Q increased less rapidly for controlled conditions relative to conventional standard stimulation (FIG. 24) due to the adjustments in PW below the maximum value in both controllers and the low duty cycle employed with C-Cont. Because of these lower Q values producing the same, if not more, work with controlled conditions compared with conventional stimulation, all calculated n values were found to be positive (FIG. 25). Both S-Cont and C-Cont stimulation thus produce more work per unit charge injected than S-Max.

FIG. 23 shows Example (LEFT) standard controller (P04) and (RIGHT) carousel controller (P02) PW output over time. Each color indicates PW delivered through an independently-controlled electrode contact while active. Gaps in delivered PWs correspond to times when each contact is inactive within the cycling scheme. Note the right leg of P04 receives a higher PA than other contacts, prompting PW cutoff at a lower maximum of 180 us for all stimulation conditions to ensure maximum charge remained below conservative stimulus level safety thresholds.

### Physiological Outcomes: Muscle Oxygen Saturation and Heart Rate

Muscle oxygen saturation (SmO2) was measured for three participants during comparisons of S-Max and S-Cont stimulation (FIG. 26). Cycling induced the largest decreases in SmO2 for participant P04, declining from approximately 75-80% in all muscles at baseline to approximately 10% within the first 20 seconds of S-Max stimulation. In contrast, SmO2 in that same participant did not decline to those levels until 75 and 120 seconds for the Left RF and Left VL respectively with S-Cont stimulation. Cycling resulted in much less drastic declines in SmO2 for participants P05 and P06, with values steadily at or above 65-70% in most muscles for both S-Max and S-Cont. Even still, SmO2 did tend to be maintained at slightly higher values with S-Cont stimulation compared with S-Max in these participants as well.

FIG. 23 shows charge accumulation for each controlled stimulation condition for participants with (LEFT) multiple independent stimulation channels and (RIGHT) a single stimulation channel. Note differences in y-axes scale. Filled circles represent total Q by the end of each participant's respective trial times (vertical dotted lines). All feedback-controlled stimulation paradigms inject less charge compared with S-Max stimulation. S-Cont data unavailable for P01, P02, and P03 and C-Cont data unavailable for P03 due to at-home data collection with standalone ECUs.

FIG. 25 shows the difference in average stimulation efficiency compared with S-Max stimulation for each participant and test condition. Positive differences indicate controlled stimulation paradigms result in more cycling output per unit charge injected.

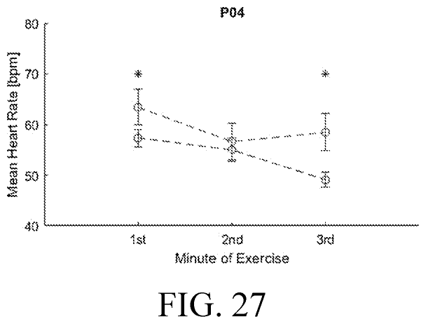

Heart rate was also monitored during select trials for P04, who demonstrated the greatest improvement in cycling performance (W and Pend) and SmO2 profiles (Left LV and Left RF) with S-Cont stimulation. S-Cont produced significantly greater (p<0.01) heart rates throughout the first and third minute of the participant's 3-minute cycling trials (FIG. 27). Heart rate increased from averages of 57 to 63 bpm in the first minute and from 49 to 58 bpm in the third minute. Interestingly, heart rate was higher though not significantly different than S-Max during the second minute of exercise, with averages of and 55 and 57 bpm for S-Max and S-Cont respectively.

FIG. 26 shows mean muscle oxygenation (SmO2) throughout S-Max and S-Cont cycling trials for P04, P05, and P06. Shaded regions represent standard deviations.

### Discussion

### Cycling Endurance

Endurance was significantly improved in five out of six participants, as evident by higher Pend values, by at least one of the cadence-controlled stimulation conditions relative to conventional stimulation. S-Cont stimulation significantly improved Pend by at least 13.5% in four of the six participants tested, with the other two participants showing positive, though non-significant improvements as well. Of the participants who cycled with C-Cont stimulation, all three exhibited significant Pend increases of at least 21.7%. Improved end power is important as it can enable users to extend exercise durations prior to lower extremity muscular fatigue, which may increase benefits to those muscles and provide a better cardiovascular workout.

FIG. 27 shows P04 heart rate responses during S-Max and S-Cont stimulation-induced cycling bouts. Heart rates are averaged over the first, second, and third minutes of the cycling trials (p<0.05).

Target cadence was always selected such that the corresponding power output on a chosen gear at that cadence can result in a mid-level exercise intensity in between the peak and end/steady state power can achieved with S-Max. Ideally, target cadence and corresponding mid-level power output can be maintained throughout the entire exercise duration. However, this was not always the case. It cannot not always be guaranteed that a participant can maintain a chosen, often quite challenging, target output that was greater than their typical steady state cycling abilities. Nevertheless, even when target cadences were not maintained throughout the entire trial due to controllers being unable to recruit any additional fibers once reaching maximum stimulus levels, power output still consistently remained above that of conventional stimulation in the final third of the trials. This indicates energy savings attained in the beginning of controlled trials could be successfully harnessed later, even once stimulation intensities eventually matched that of conventional stimulation.

One participant in particular, P03, showed remarkable percent improvements in Pend with cadence-controlled stimulation (297% and 867% for S-Cont and C-Cont respectively). This participant initially output powers up to 30 watts, but was unable to cycle beyond, and sometimes even up to, 90 seconds continuously with the S-Max pattern. P03's power output can decline so swiftly that pedal revolutions could not occur unassisted within this very short period of time. Power readings often declined to 0 watts with conventional stimulation before the trial duration was over. With both controlled stimulation conditions, P03 was able to more consistently pedal throughout the entire 90 second trial duration, leading to the large percent increases and solidifying the endurance benefits of these controlled stimulation approaches.

Only two participants did not gain statistically significant improvements in Pend using S-Cont stimulation. P02 has only completed two trials of this condition thus far due to a temporary suspension of remote data collection. So, while the difference is trending positively, there are not enough samples to reach statistical significance. Additionally, the target cadence chosen for these two trials produced a power only slightly above this participant's steady state, which was easily maintained with the controller throughout the entire 2.5 minute cycling duration. P02 could have cycled at the chosen target well beyond 2.5 minutes, and that a more challenging target choice in the future can still be sustainable for that duration and produce greater, more significant improvements over conventional stimulation. P05, however, is perhaps the most consistent cyclist with the best baseline endurance and smallest range of peak to steady state power out of all participants, reporting steady cycling within a 4-5 rpm window for over an hour on his home setup using conventional stimulation. Because P05 already has excellent endurance and only an approximately 3 Watt range in which to find a mid-level target, there was little room for the controller to work and show improvements over conventional stimulation. For this participant, cadence control is unlikely to provide large benefits at their current ability level. Focus can instead be directed towards increasing P05's absolute strength, as cycling durations were long but power outputs remained low throughout, preventing cycling against meaningful resistances.

### Exercise Intensity

Exercise intensity is indicated by the total work performed within a set cycling duration. Only one participant significantly increased work performed over conventional stimulation using S-Cont. Similarly, only two of the three tested participants performed significantly more work with the C-Cont scheme. Work is calculated as area under the power curve; therefore, target cadence choice and the resultant power output value strongly affects how quickly work will accumulate. As stated above, targets were chosen to produce and maintain a power between the peak and steady state power achieved with conventional stimulation in order to extend power output and cycling duration. They were not necessarily chosen to ensure maintenance of the resulting power leads to equal or greater work performed within the same cycling duration. Even still, the fact that work did accumulate to a significantly greater degree in three participants is encouraging. It is likely that conditions resulting in significantly improved end power in other participants could eventually accumulate further improvements in work if trial durations were extended.

### Power Fluctuation and Pedaling Smoothness

Power fluctuation indices were measured to assess pedaling smoothness. Prior studies have found that uneven force production among independently activated fiber groups is a significant practical limitation of stimulation patterns that attempt to reduce duty cycle. A recent investigation in the laboratory showed incorporating only the strongest MUPs in a low duty cycle scheme could significantly reduce this power fluctuation over approaches that attempted to include additional weaker pools, but still resulted in significantly higher fluctuations compared with conventional stimulation. The C-Cont stimulation pattern presented here not only provided the benefits of reduced duty cycle, but also allowed precise control of each independently activated fiber group by its own PI controller. Controlling only one stimulating contact per pedal stroke ensured that stimulation was adjusted precisely according to the needs of that fiber group to evoke even force outputs each pedal rotation. C-Cont successfully reduced PFI to the point of no significant difference with S-Max in two of three participants tested. PFI remained significantly higher than both S-Max and S-Cont in the third participant, but was still considerably lower than PFIs from open-loop carousel stimulation studies. While the PFI difference was statistically significant, absolute power deviations were well tolerated and did not discourage P03 from cycling with this pattern. Results of this study thus provide a way to practically employ duty cycle reducing cycling patterns without the limitations of open-loop implementation to better improve cycling ability.

In four of six participants, S-Cont resulted in no significant change in PFI compared with S-Max. This is as expected since every fiber pool is activated each pedal rotation and uneven output from independently activated groups is not an issue. Unexpectedly, though, S-Cont did significantly increase PFI in two participants. P01 was utilizing an earlier version of controller code compiled to the standalone ECU during home experiments that was only later found to have to processing inefficiencies which at times produced erratic controller behavior. That original standalone controller model was since recoded to lower processing times and improve tracking performance. This new code is what was used for the remainder of the at-home S-Cont experiments performed by P02 and P03, which did demonstrate PFIs more comparable to S-Max. Therefore, it is contemplated that the higher PFI found with S-Cont to resolve should P01 be retested with the newer model. This may even have a greater impact on P01's accumulated work and power maintenance as well.

The standard controller adjusts stimulation through all active contacts at once, delivering the same PW through each. This means the controlled PW is essentially operating along the combined recruitment curve (RC) that results from stimulating all contacts with equal PWs at once. Independent MUPs activated by individual contacts will sum approximately linearly when there is no overlap in stimulated fibers. The combined RC then is likely very steep, especially at lower stimulus values, such that a small change in stimulus level corresponds with a large change in muscle output. P04, as the strongest participant of the group, may have the steepest increase in power output per change in PW. A steep RC will make finding an exact PW value for target cadence maintenance more difficult especially as the fibers contributing to that curve potentiate and fatigue at different rates. This likely increased the difficulty for the controller to identify the ideal PWs in P04, resulting in increased PFI. This signifies a need for improving controller performance, especially for the strongest and most fatigable participants, which is addressed further in a later section.

### Physiological Effects of Controlled Stimulation

Physiological muscle oxygenation data collected with three participants during S-Max and S-Cont trials may explain variations in functional performance among participants. In P04, who received significant functional advantages from S-Cont compared with S-Max (9.4% more work, 21.6% higher end power), drastic, sudden declines in SmO2, from approximately 80% at baseline to less than 10% within the first 20 seconds of pedaling, in all three heads of the quadriceps with S-Max stimulation were observed. S-Cont showed much slower SmO2 declines in P04, particularly in the rectus femoris and vastus lateralis fiber groups which only reached the same low steady state after 80 and 120 seconds respectively. It is possible that this subject has a previously undetected perfusion issue that causes activated fibers to consume oxygen much faster than it is supplied. By delaying the incorporation of some fibers until needed with S-Cont, oxygen supplies are not used up immediately in all fibers. This may enable more efficient aerobic respiration to occur in some parts of the muscles longer than with S-Max, and account for the significant functional improvements seen. In the other two participants, however, conventional stimulation did not induce such drastic SmO2 declines, dropping approximately 30% at most. Much less dramatic improvements in oxygenation were therefore observed with S-Cont, which may explain why significant differences in cycling performance were not seen between stimulation conditions with P05 and P06.

It should also be noted that P04 was training for the 2020 Cybathlon during the period of data collection. P04's baseline strength was much higher than the other two subjects on which muscle oxygenation tests were performed. Very high baseline power output enabled P04 to cycle on a much harder gear and thus against much greater resistances than P05 and P06. It is possible that pedaling against greater resistances contributed to the quick and dramatic decline in SmO2 seen with conventional stimulation, providing ample opportunity for improvement with the controller. It is possible that, should the other participants be able to increase resistances as well, the controller can become more beneficial to them when the demand on the muscles in more extreme and better pacing strategies become more valuable. Future work can assess the relative rates of SmO2 decline at various gear and cadence combinations to determine a combination that enables high powers to be maintained with the physiological advantage of slower oxygenation decline.

A significant heart rate increase when cycling with cadence-controlled stimulation is another notable physiological benefit seen in P04. Paralysis, particularly when caused by SCI, hinders the cardiorespiratory systems' ability to appropriately respond to stimulated exercise. Loss of quick afferent feedback from the working muscles to the autonomic nervous system because of the lesion suppresses the exercise pressor reflex, which is responsible for the immediate and appropriate regulation of heart rate, respiration rate, and blood pressure during exertion. Observations in the laboratory have revealed heart rate often changes only negligibly and sometimes even declines in participants with SCI despite cycling to the point of lower extremity exhaustion. A reduction in heart rate seen in people with SCI is due to increased venous return when the typically sedentary lower extremities are activated by stimulation. Contraction of the paralyzed muscles creates a pumping effect that can greatly increase blood volume returned to the heart. Increased blood volume means the heart does not have to beat as fast to maintain the same cardiac output, so heart rate is depressed despite actually needing to be increased. These factors prevent conventional stimulation-induced cycling from providing a meaningful cardiovascular workout and present obstacles for providing the working muscles with the resources they need to keep moving, which may additionally contribute to rapid fatigue.

The fact that cadence-controlled stimulation was able to overcome these barriers to elevating heart rate in P04 is extremely promising. Because the controller does not maximally stimulate to recruit all available fibers from the outset of the exercise and maintains a lower than maximum cadence, venous return may have changed less drastically than in conventional stimulation due to only a subsection of the quadriceps pumping at slower rates. This may have initially eased the blood volume-induced heart rate depression phenomena, enabling greater heart rates to be maintained in the first minute of exercise. Greater accumulation of work performed by P04 with cadence-controlled stimulation may have enabled enough non-neural, blood-borne factors to trigger positive heart rate increases and overcome the heart rate suppression in the third minute. The statistically significant increases in heart rate during the first and third minute of exercise, while only 6-9 beats per minute, may have facilitated greater oxygen delivery to the quadriceps muscles to help sustain power and perform even more work, perpetuating the cycle.

### Controller Performances

Maintaining a submaximal target cadence with controlled stimulation enabled lower level stimuli to recruit a smaller number of fibers from the outset of exercise. As shown in FIG. 23, both S-Cont and C-Cont patterns dynamically adjusted PW through each quadriceps-activating contact to account for both potentiation and fatigue of the independent MUPs. When the originally recruited fibers became unable to maintain the desired cycling output, the controller increased stimulation intensity only as much as was necessary to recruit more unfatigued fibers to assist in cycling. Controlled patterns therefore delayed the incorporation of all available knee extensor fibers, enabling energy to be reserved for later use instead of exhausting all fibers at once as in conventional S-Max stimulation.

Because of these adjustments in PW below the maximum, Q accumulated to a much lesser degree at the end of all available trials with the controlled conditions. Difference in Q is most drastic for the carousel controller conditions as submaximal stimulation is only being delivered through a single contact each pedal stroke. From the available in-laboratory data, cadence controlled cycling patterns were all found to be more efficient than conventional stimulation, producing more work per unit of charge injected. Efficiency was even increased for those participants and conditions where work was not found to be significantly higher, due to the very low levels of Q needed to sustain the lower target output demand. Higher efficiency is crucial for extending battery life of the ECUs so that participants may cycle and still use their neuroprostheses in other applications throughout the day. Reducing the amount of charge necessary to maintain a desired exercise intensity provides further assurance that no overstimulation or damage to the neural tissue will result over time. Additionally, prior research demonstrates a strong correlation between stimulation cost, the inverse of stimulation efficiency presented here, and the oxygen cost (the rate of pulmonary oxygen uptake) of an exercise. Greater stimulation efficiency during controlled stimulation conditions from this study may therefore coincide with less oxygen cost, which can make maintaining a given exercise intensity less taxing on a participant's pulmonary system. Though not formally measured in this study, several participants, particularly P04, did anecdotally report feeling less breathless after controlled cycling bouts compared with conventional stimulation. Further work is needed to formally validate the relationship between stimulation efficiency and metabolic cost, but if correlations remain consistent, stimulation efficiency may be an easier measurement of exercise efficacy that removes the need for participants to wear gas exchange sensors and improves accuracy by removing noise from breath-by-breath gas exchange variations.

The control schemes used in this study were originally designed for simplicity so that they may be successfully compiled into portable ECUs for eventual overground use. The focus was not necessarily on creating a controller with minimal rise time and maximum disturbance rejection, but rather on approximating a mid-level target with minimal processing time. Nevertheless, implementation of basic PI control yielded average absolute RMSE across all participants of only 2.4 rpm (6.4%). More sophisticated control schemes have been proposed from simulations with musculoskeletal models that may more faithfully produce the desired output with quicker rise times, less overshoot, and minimal deviations from the target. However, these proposed cadence control schemes have not yet been successfully deployed in clinical tests with paralyzed subjects or without simultaneous, prioritized control of a motor. The need for an accurate and individualized musculoskeletal model and significant training times remain prominent barriers to their practical implementation. Still, future work can seek to incorporate these more advanced control schemes into a motorless stimulation system to potentially provide even greater improvements in exercise performance and physiological outcomes.

### Advantages of Motorless Exercise Control

Prior studies incorporating feedback control during stimulation-induced cycling utilized a motor to provide assistance or resistance against stimulated muscle contractions to maintain a target output. These approaches have the advantage of providing greater resistance in the beginning of a trial to maximize the load against which the muscle must work before it is fatigued. Maximizing exercise intensity with resistive loads may be the key to achieving greater load-dependent physiologic improvements with these systems, especially in bone density. However, use of a motor also comes with numerous disadvantages. Excessive motor resistance may prematurely fatigue the muscles and drastically reduce the duration of exercise. Assisting the pedaling motion when muscle output becomes insufficient for target maintenance can shield paralyzed musculature from positive stress and decrease required effort that can help them improve. There are mixed opinions as to whether keeping the legs cycling after the muscles can no longer contribute to the motion provides adequate physiological benefits, and the fatigued muscles may be better served resting without continued ineffective stimulation so that they may recover and perform subsequent bouts of meaningful, leg-driven (as opposed to motor-driven) cycling. Additionally, integrating a motor significantly increases the complexity of the control algorithm to avoid potential harm to the participant, as well as the weight and cost of the cycling apparatus. Control methods from this study are easily implemented with no additional hardware or musculoskeletal modeling requirements and, after refining, only minute increases in computational complexity. This makes them ideal for practical every day and potentially overground use. Maintaining a mid-level intensity using only the capabilities of the activated muscles ensures that meaningful work against resistive loads is performed without undue fatigue or assistance.

Based on these results, it can be understood that that charge accumulation was lower than conventional supramaximal stimulation since initial PW conditions were set to be submaximal and a submaximal cadence was maintained at least temporarily in all trials. This can further translate to assumed improved efficiencies since work was unchanged or even somewhat improved in all at-home trials as well.

In exemplary aspects, the disclosed system can provide a range of user-adjusted target options. Cyclists can then have the freedom to adjust based on their desired exercise time and may be able to adapt exercise intensities as their fitness level changes over time.

### CONCLUSIONS

Cadence control of neural stimulation intensity successfully extended cycling endurance in a motorless system. Both standard and duty cycle reducing control schemes were found to prolong activated muscle output compared to conventional stimulation techniques by maintaining a mid-level exercise intensity. Extending exercise durations without interference of a motor can allow participants with paralysis to obtain greater physiological benefits, as demonstrated by preliminary heart rate and muscle oxygen saturation measurements that improved significantly with cadence control. Though significant increases in work were only found in three participants, significantly higher power maintenance at the end of controlled trials may enable significantly more work to accumulate with increased trial durations in all participants. Finally, simple control schemes used in this study provided stable power output and good target cadence tracking performance with minimal processing and no training or modeling required, making them suitable for widespread, practical use with the potential to enable overground cycling.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A vehicle that is movable along a surface, the vehicle comprising:
a plurality of wheels;
a propulsion assist system comprising:
   at least one battery;
   a motor that is operatively coupled to at least one wheel of the plurality of wheels and configured to cause rotation of the at least one wheel of the plurality of wheels;
   a controller in electrical communication with the motor; and
   at least one orientation sensor in communication with the controller, wherein the at least one orientation sensor is configured to determine a sensed orientation of the vehicle,
   wherein the controller is configured to modulate a power output of the motor based at least in part on the sensed orientation of the vehicle.

Aspect 2: The vehicle of aspect 1, wherein the vehicle has a front portion, a rear portion, and a longitudinal axis that extends between the front portion and the rear portion of the vehicle, wherein the sensed orientation comprises an orientation of the longitudinal axis of the vehicle relative to a horizontal plane.

Aspect 3: The vehicle of aspect 1 or aspect 2, wherein the at least one orientation sensor comprises an inertial measurement unit.

Aspect 4: The vehicle of any one of the preceding aspects, wherein the at least one orientation sensor comprises a 3-axis accelerometer, a 3-axis gyroscope, and a 3-axis magnetometer.

Aspect 5: The vehicle of any one of the preceding aspects, wherein the controller is further configured to, based on feedback from the at least one orientation sensor, determine a terrain condition, wherein the controller is configured to modulate a power output of the motor at least in part based on the terrain condition.

Aspect 6: The vehicle of aspect 5, wherein the controller is further configured to perform a fast Fourier transform (FFT) on the feedback from the at least one orientation sensor to determine a terrain condition.

Aspect 7: The vehicle of aspect 5, wherein the terrain condition comprises terrain roughness.

Aspect 8: The vehicle of any one of the preceding aspects, wherein the vehicle is a cycling device comprising:
a crankset in communication with at least one wheel of the plurality of wheels;
a pair of appendage receptacles that are coupled to the crankset.

Aspect 9: The vehicle of aspect 8, wherein the pair of appendage receptacles are each configured to immobilize a respective joint of a user.

Aspect 10: The vehicle of aspect 8, wherein the cycling device is a recumbent tricycle.

Aspect 11: The vehicle of any one of the preceding aspects, wherein the vehicle is a wheelchair.

Aspect 12: The vehicle of any one of the preceding aspects, wherein the motor is a brushless motor.

Aspect 13: The vehicle of any one of the aspects 1-11, wherein the motor is a brushed motor.

Aspect 14: The vehicle of any one of the preceding aspects, wherein the motor is configured to apply a torque to the at least one wheel in a rotational direction that corresponds to forward movement of the vehicle.

Aspect 15: The vehicle of any one of the preceding aspects, wherein the motor is configured to apply a torque to the at least one wheel in a rotational direction that resists forward movement of the vehicle.

Aspect 16: The vehicle of any one of the preceding aspects, wherein the controller is configured to determine a crankset position based on feedback from the at least one orientation sensor.

Aspect 17: A method comprising:
sensing, by at least one orientation sensor, an incline of a vehicle as in any one of aspects 1-16; and
controlling a power output of a motor based at least in part on the incline of the vehicle, wherein the motor is operatively coupled to at least one wheel of the vehicle.

Aspect 18: The method of aspect 17, further comprising:
sensing, by the at least one orientation sensor, a terrain condition upon which the vehicle is traveling;
controlling the power output of the controller based at least in part on the terrain condition.

Aspect 19: A system comprising:
a cycling device comprising:
   a crankset;
   a pair of appendage receptacles that are coupled to the crankset, wherein the pair of appendage receptacles are each configured to immobilize a respective joint of a user;
   a crankset angle sensor coupled to the crankset, wherein the crankset angle sensor is configured to provide an output indicative of an angle of the crankset; and
a controller in communication with the crankset angle sensor;
wherein the controller is configured to control stimulation from an external or implanted pulse generator based at least in part on the angle of the crankset.

Aspect 20: The system of aspect 19, wherein the controller comprises a wireless receiver, wherein the cycling device further comprises a wireless transmitter that is in communication with the crankset angle sensor, wherein the controller is in wireless communication with the crankset angle sensor by the wireless transmitter.

Aspect 21: The system of aspect 19 or aspect 20, wherein the controller is in wired communication with the crankset angle sensor.

Aspect 22: The system of any one of aspects 19-21, wherein the crankset angle sensor comprises a rotary encoder.

Aspect 23: The system of aspect 23, further comprising a transmission, wherein the transmission comprises one of:
a direct coupling between the rotary encoder and the crankset;
a first pulley that is rotationally fixed to the crankset, a second pulley that is coupled to the rotary encoder, and a belt that extends between the first pulley and the second pulley; or
a first gear that is coupled to the rotary encoder and a second gear that is coupled to the crankset, wherein the first gear is coupled to the second gear.

Aspect 24: The system of any one of aspects 19-24, wherein the crankset angle sensor comprises at least one orientation sensor that is coupled to the crankset.

Aspect 25: The system of aspect 24, wherein the at least one orientation sensor comprises an inertial measurement unit.

Aspect 26: The system of aspect 24 or aspect 25, wherein the at least one orientation sensor comprises a plurality of orientation sensors.

Aspect 27: The system of any one of aspects 19-26, further comprising an external or implanted pulse generator in communication with the controller.

Aspect 28: The system of aspect 27, wherein the functional neural stimulation apparatus comprises a plurality of electrodes that are configured to stimulate respective muscles of the user.

Aspect 29: The system of any one of aspects 19-28, wherein the controller is configured to deliver functional neural stimulation to a plurality of groups of muscle fibers, wherein, for each group of muscle fibers, stimulation is configured to start at a respective first rotational position of the crankset and cease at a respective second rotational position of the crankset.

Aspect 30: The system of any one of aspects 19-29, further comprising a computing device in communication with the controller, wherein the computing device is configured to:
provide an interface to a clinician;
receive, by the interface, at least one parameter selection from the clinician; and
set at least one control parameter of the controller.

Aspect 31: The system of aspect 30, wherein the at least one control parameter comprises at least one of a stimulation current, a pulse width, a start angle corresponding to an angle of the crankset at which stimulation begins, or a stop angle corresponding to an angle of the crankset at which stimulation ceases, wherein each control parameter of the at least one control parameter is associated with a particular group of fibers of muscle fibers.

Aspect 32: The system of any one of aspects 19-31, wherein the cycling device comprises:
a plurality of wheels, wherein the crankset is coupled to at least one wheel of the plurality of wheels.

Aspect 33: The system of aspect 32, wherein the cycling device is a recumbent tricycle.

Aspect 34: The system of any one of aspects 19-33, wherein the cycling device is a stationary bike.

Aspect 35: The system of any one of aspects 19-34, further comprising a display that is configured to display visual feedback associated with use of the cycling device.

Aspect 36: The system of aspect 35, wherein the display is a virtual reality device or an augmented reality device.

Aspect 37: The system of any one of aspects 19-36, further comprising at least one respiration measurement device.

Aspect 38: The system of any one of aspects 19-37, further comprising at least one grip sensor.

Aspect 39: A method comprising:
cyclically stimulating fibers of a plurality of muscles of a user having at least one appendage comprising a distal portion, wherein the distal portion of the at least one appendage of the user is coupled to a crankset, wherein cyclically stimulating the fibers of the plurality of muscles of the user comprises beginning stimulation of the fibers of each muscle of the plurality of muscles at a respective first angle of the crankset and ceasing stimulation of the fibers of each muscle of the plurality of muscles at a respective second angle of the crankset.

Aspect 40: The method of aspect 39, wherein the plurality of muscles comprises two or more of: a left quadriceps, a left gluteus maximus, a left hamstring extensor, a left hamstring flexor, a right quadriceps, a right gluteus maximus, a right hamstring extensor, or a right hamstring flexor.

Aspect 41: The method of aspect 40, wherein the plurality of muscles comprises each of: the left quadriceps, the left gluteus maximus, the left hamstring extensor, the left hamstring flexor, the right quadriceps, the right gluteus maximus, the right hamstring extensor, and the right hamstring flexor.

Aspect 42: The method of any one of aspects 39-41, further comprising:
measuring an exercise metric;
comparing the exercise metric to a target exercise metric; and
modifying at least one stimulation parameter based on the exercise metric.

Aspect 43: The method of aspect 42, wherein the exercise metric is a heart rate, wherein the target exercise metric is a target heart rate.

Aspect 44: The method of aspect 42, wherein the exercise metric is a ventilation rate and the target exercise metric is a target ventilation rate

Aspect 45: The method of aspect 42, wherein the exercise metric is a power output, wherein the target exercise metric is a target power output.

Aspect 46: The method of aspect 42, wherein the exercise metric is a crankset rotation speed, wherein the target exercise metric is a target crankset rotation speed.

Aspect 47: The method of aspect 42, wherein modifying the at least one stimulation parameter comprises increasing or decreasing at least one parameter to increase or decrease the exercise metric toward the target exercise metric.

Aspect 48: The method of aspect 47, wherein the at least one stimulation parameter comprises at least one of a pulse width, a stimulation current, an angle of the crankset at which stimulation begins, or a stop angle corresponding to an angle of the crankset at which stimulation ceases.

Aspect 49: The method of any one of aspects 42-48, wherein modifying the at least one stimulation parameter comprises modifying the at least one parameter based on machine learning.

Aspect 50: The method of aspect 49, wherein the machine learning comprises one of iterative learning control or reinforcement learning control.

Aspect 51: The method of any one of aspects 42-50, further comprising receiving the target exercise metric from a clinician or the user.

Aspect 52: The method of aspect 51, wherein receiving the target exercise metric comprises receiving the target exercise metric during an exercise session.

Aspect 53: The method of any one of aspects 39-52, further comprising displaying on a display at least one visual element associated with exercise generated by stimulation of the fibers of the plurality of muscles.

Aspect 54: The method of aspect 53, wherein the display is an augmented reality display or a virtual reality display.

Aspect 55: The method of any one of aspects 39-54, further comprising:
receiving a volitional effort input from the user; and
displaying, on the display, a metric associated with the volitional effort input.

Aspect 56: The method of aspect 55, wherein the volitional effort input is a force or pressure sensor associated with grip.

Aspect 57: The method of any one of aspects 39-56, further comprising measuring electromyography signals of the user.

Aspect 58: The method of any one of aspects 39-57, further comprising:
measuring respiration of the user; and
displaying measured respiration of the user.

Aspect 59: The method of any one of aspects 39-58, wherein the crankset is a portion of a stationary bike.

Aspect 60: The method of any one of aspects 39-59, wherein the crankset is a portion of a cycling device, wherein the cycling device comprises a plurality of wheels, wherein the crankset is coupled to at least one wheel of the plurality of wheels.

Aspect 61: The method of aspect 60, wherein the cycling device is a recumbent tricycle.

Aspect 62: A method comprising:
stimulating a first portion of a first muscle of a user during a first cycle of an exercise; and
stimulating a second portion of the first muscle of the user during a second cycle of the exercise.

Aspect 63: The method of aspect 62, wherein the exercise is rowing.

Aspect 64: The method of aspect 62 or aspect 63, wherein the exercise is cycling.

Aspect 65: A method comprising:
measuring, continually or iteratively, positions of an exercise apparatus along a circuit, wherein the exercise apparatus is configured for cyclic movement along the circuit; and
cyclically stimulating a plurality of muscles of a user coupled to the exercise apparatus based on the position of the exercise apparatus.

Aspect 66: The method of aspect 65, wherein the exercise apparatus is a rowing machine, wherein measuring, continually or iteratively, the positions of the exercise apparatus along the circuit comprises using a linear position sensor to measure the positions of the exercise apparatus along the circuit.

Aspect 67: The method of aspect 65 or aspect 66, wherein the exercise apparatus is a stationary bike or an elliptical trainer.

Aspect 68: The method of any one of aspects 65-67, wherein the exercise apparatus is a cycling vehicle.

Aspect 69: The method of any one of aspects 65-68, further comprising:
measuring an exercise metric;
comparing the exercise metric to a target exercise metric; and
modifying at least one stimulation parameter based on the exercise metric.

Aspect 70: The method of aspect 69, wherein the exercise metric is a heart rate, wherein the target exercise metric is a target heart rate.

Aspect 71: The method of aspect 69, wherein the exercise metric is a ventilation rate and the target exercise metric is a target ventilation rate

Aspect 72: The method of aspect 69, wherein the exercise metric is a power output, wherein the target exercise metric is a target power output.

Aspect 73: The method of aspect 69, wherein the exercise metric is a circuit completion speed, wherein the target exercise metric is a target circuit completion speed.

Aspect 74: The method of any one of aspects 65-73, wherein modifying the at least one stimulation parameter comprises increasing or decreasing at least one parameter to increase or decrease the exercise metric toward the target exercise metric.

Aspect 75: The method of aspect 74, wherein the at least one stimulation parameter comprises at least one of a pulse width, a stimulation current, a first angle of at least one muscle, or a second angle of at least one muscle.

Aspect 76: The method of any one of aspects 65-76, wherein modifying the at least one stimulation parameter comprises modifying the at least one parameter based on machine learning.

Aspect 77: The method of aspect 76, wherein the machine learning comprises one of iterative learning control or reinforcement learning control.

Aspect 78: The method of any one of aspects 65-77, further comprising receiving the target exercise metric from a clinician or the user.

Aspect 79: The method of aspect 78, wherein receiving the target exercise metric comprises receiving the target exercise metric during an exercise session.

Aspect 80: The method of any one of aspects 65-79, further comprising displaying on a display at least one visual element associated with exercise generated by stimulation of the plurality of muscles.

Aspect 81: The method of aspect 80, wherein the display is an augmented reality display or a virtual reality display.

Aspect 82: The method of any one of aspects 65-81, further comprising:
receiving a volitional effort input from the user; and
displaying, on the display, a metric associated with the volitional effort input.

Aspect 83: The method of aspect 82, wherein the volitional effort input is a force or pressure sensor associated with grip.

Aspect 84: The method of any one of aspects 65-83, further comprising measuring electromyography signals of the user.

Aspect 85: The method of any one of aspects 65-84, further comprising:
measuring respiration of the user; and
displaying measured respiration of the user.

Although the foregoing invention has been described in some detail by way of illustration and example for purposes of clarity of understanding, certain changes and modifications may be practiced within the scope of the appended claims.

1. A vehicle that is movable along a surface, the vehicle comprising:
a plurality of wheels;
a propulsion assist system comprising:
at least one battery;
a motor that is operatively coupled to at least one wheel of the plurality of wheels and configured to cause rotation of the at least one wheel of the plurality of wheels;
a controller in electrical communication with the motor; and
at least one orientation sensor in communication with the controller, wherein the at least one orientation sensor is configured to determine a sensed orientation of the vehicle,
wherein the controller is configured to modulate a power output of the motor based at least in part on the sensed orientation of the vehicle.

2. The vehicle of claim 1, wherein the vehicle has a front portion, a rear portion, and a longitudinal axis that extends between the front portion and the rear portion of the vehicle, wherein the sensed orientation comprises an orientation of the longitudinal axis of the vehicle relative to a horizontal plane.

3. The vehicle of claim 1, wherein the at least one orientation sensor comprises an inertial measurement unit.

4. The vehicle of claim 1, wherein the at least one orientation sensor comprises a 3-axis accelerometer, a 3-axis gyroscope, and a 3-axis magnetometer.

5. The vehicle of claim 1, wherein the controller is further configured to, based on feedback from the at least one orientation sensor, determine a terrain condition, wherein the controller is configured to modulate a power output of the motor at least in part based on the terrain condition.

6. The vehicle of claim 5, wherein the controller is further configured to perform a fast Fourier transform (FFT) on the feedback from the at least one orientation sensor to determine a terrain condition.

7. (canceled)

8. The vehicle of claim 1, wherein the vehicle is a cycling device comprising:
a crankset in communication with at least one wheel of the plurality of wheels;
a pair of appendage receptacles that are coupled to the crankset.

9. The vehicle of claim 8, wherein the pair of appendage receptacles are each configured to immobilize a respective joint of a user.

10. The vehicle of claim 8, wherein the cycling device is a recumbent tricycle.

11. The vehicle of claim 1, wherein the vehicle is a wheelchair.

12. (canceled)

13. (canceled)

14. (canceled)

15. (canceled)

16. The vehicle of claim 1, wherein the controller is configured to determine a crankset position based on feedback from the at least one orientation sensor.

17. (canceled)

18. (canceled)

19. A system comprising:
a cycling device comprising:
a crankset;
a pair of appendage receptacles that are coupled to the crankset, wherein the pair of appendage receptacles are each configured to immobilize a respective joint of a user;
a crankset angle sensor coupled to the crankset, wherein the crankset angle sensor is configured to provide an output indicative of an angle of the crankset; and
a controller in communication with the crankset angle sensor:
wherein the controller is configured to control stimulation from an external or implanted pulse generator based at least in part on the angle of the crankset.

20. (canceled)

21. (canceled)

22. The system of claim 19, wherein the crankset angle sensor comprises a rotary encoder.

23. The system of claim 22, further comprising a transmission, wherein the transmission comprises one of:
a direct coupling between the rotary encoder and the crankset:
a first pulley that is rotationally fixed to the crankset, a second pulley that is coupled to the rotary encoder, and a belt that extends between the first pulley and the second pulley: or
a first gear that is coupled to the rotary encoder and a second gear that is coupled to the crankset, wherein the first gear is coupled to the second gear.

24. (canceled)

25. (canceled)

26. (canceled)

27. The system of claim 19, further comprising an external or implanted pulse generator in communication with the controller.

28. (canceled)

29. The system of claim 19, wherein the controller is configured to deliver functional neural stimulation to a plurality of groups of muscle fibers, wherein, for each group of muscle fibers, stimulation is configured to start at a respective first rotational position of the crankset and cease at a respective second rotational position of the crankset.

30. The system of claim 19, further comprising a computing device in communication with the controller, wherein the computing device is configured to:
provide an interface to a clinician;
receive, by the interface, at least one parameter selection from the clinician; and
set at least one control parameter of the controller.

31. (canceled)

32. The system of claim 19, wherein the cycling device comprises:
a plurality of wheels, wherein the crankset is coupled to at least one wheel of the plurality of wheels.

33. (canceled)

34. (canceled)

35. The system of claim 19, further comprising a display that is configured to display visual feedback associated with use of the cycling device.

36. (canceled)

37. The system of claim 19, further comprising at least one respiration measurement device.

38. The system of claim 19, further comprising at least one grip sensor.

39. A method comprising:
cyclically stimulating fibers of a plurality of muscles of a user having at least one appendage comprising a distal portion, wherein the distal portion of the at least one appendage of the user is coupled to a crankset, wherein cyclically stimulating the fibers of the plurality of muscles of the user comprises beginning stimulation of the fibers of each muscle of the plurality of muscles at a respective first angle of the crankset and ceasing stimulation of the fibers of each muscle of the plurality of muscles at a respective second angle of the crankset.

40-85. (canceled)
